(19) United States
(12) Patent Application Publication
(10) Pub. No.: US 2024/0175867 A1
(43) Pub. Date: May 30, 2024

(54) METHODS OF GRAFT VERSUS HOST DISEASE DIAGNOSIS

(71) Applicants: HADASIT MEDICAL RESEARCH SERVICES AND DEVELOPMENT LTD., Jerusalem (IL); YISSUM RESEARCH DEVELOPMENT COMPANY OF THE HEBREW UNIVERSITY OF JERUSALEM LTD., Jerusalem (IL)

(72) Inventors: Batia AVNI, Ness Ziona (IL); Benjamin GLASER, Jerusalem (IL); Yuval DOR, Jerusalem (IL); Ruth SHEMER, Mevasseret Zion (IL)

(21) Appl. No.: 18/389,293

(22) Filed: Nov. 14, 2023

Related U.S. Application Data
(60) Provisional application No. 63/428,778, filed on Nov. 30, 2022.

(51) Int. Cl. G01N 33/53; G01N 33/68

(52) U.S. Cl. CPC G01N 33/5308; G01N 33/6863; G01N 2800/245

(57) ABSTRACT

Methods of diagnosing graft versus host disease (GVHD) comprising quantifying cfDNA or employing a machine learning model are provided. Methods comprising training a machine learning model are also provided.

### CROSS REFERENCE TO RELATED APPLICATIONS

This application claims the benefit of priority of U.S. Provisional Patent Application No. 63/428,778, filed Nov. 30, 2022, the contents of which are all incorporated herein by reference in their entirety.

### REFERENCE TO AN ELECTRONIC SEQUENCE LISTING

The contents of the electronic sequence listing (HDST-HUJI-P-016-US.xml; Size: 50,283 bytes; and Date of Creation: Nov. 7, 2023) is herein incorporated by reference in its entirety.

### FIELD OF INVENTION

The present invention is in the field of graft versus host disease diagnostics.

### BACKGROUND OF THE INVENTION

Hematopoietic stem cell transplantation (HCT) is an essential and often the sole curative treatment strategy for high-risk hematologic malignancies. Graft versus host disease (GVHD), the foremost complication of allogeneic HCT, is a major limitation of this procedure, accounting for deleterious effects on quality of life and increased mortality from HCT. Currently, diagnosis of acute (aGVHD) and chronic GVHD (cGVHD) in bone marrow transplant patients is based on inaccurate, operator-dependent clinical markers, and less often on biopsies. These methods are time consuming, costly, invasive and yield late-stage diagnoses that negatively affect morbidity and mortality. In addition, current practice lacks accurate biomarkers for prediction of disease occurrence, identification of disease onset, prediction of disease response to treatment and accurate assessment of the actual response to treatment. Multiple prognostic and diagnostic biomarkers for cGVHD have been proposed, including IL2Rα, aminopeptidase N (CD13), IL4, IL6, TNFα, ST2, OPN, chemokine ligands such as CXCL9, CXCL10, and CXCL11, cellular biomarkers including immune cells subpopulations, and miRNA. However, none of these biomarkers have been clinically validated. In addition, all these markers are indicative of immune system derangement, lacking information on the damaged tissue targeted by the allo-immune process. Thus, there is an unmet need for simple objective tools that can aid the treating physician in easier identification and scoring and assist personalization of management in patients suffering from GVHD.

Classic liquid biopsies analyze circulating cell-free DNA (cfDNA) via genetic variations or mutations in the DNA of a fetus, a tumor or a transplanted solid organ. However, these approaches are blind to DNA released from cells with a normal genome, as would occur in organs damaged by pathologies such as GVHD. It has been shown that tissue-specific DNA methylation patterns can provide powerful, universal biomarkers for detecting the tissue origins of cfDNA, reflective of elevated turnover or damage in specific organs and regardless of the underlying pathology. For example, it has been shown that genomic loci specifically unmethylated in lung epithelial cells or in hepatocytes can serve as cfDNA biomarkers detect specific lung or liver injury.

There is a great need for accurate biomarkers for prediction of GVHD occurrence, identification of disease onset, prediction and assessment of disease severity and response to treatment.

### SUMMARY OF THE INVENTION

The present invention provides methods of diagnosing graft versus host disease (GVHD) comprising receiving a measurement of cell-free DNA (cfDNA) quantity in a sample, wherein a cfDNA quantity above a predetermined threshold indicates the subject suffers from GVHD. Methods of diagnosing GVHD comprising applying a machine learning model to at least two parameters from a sample are also provided.

According to a first aspect, there is provided a method of diagnosing graft versus host disease (GVHD) in a subject in need thereof, the method comprising receiving a measurement of cell-free DNA (cfDNA) quantity in a fluid sample from the subject, wherein a cfDNA quantity above a predetermined threshold indicates the subject has GVHD, thereby diagnosing GVHD.

According to some embodiments, the subject underwent a hematopoietic stem cell transplant (HCT) from a donor.

According to some embodiments, the HCT occurred at least 100 days before the sample was taken.

According to some embodiments, the GVHD is chronic GVHD.

According to some embodiments, the method further comprises extracting the fluid sample from the subject, isolating cfDNA from the extracted sample and quantifying the isolated cfDNA.

According to some embodiments, the fluid is selected from peripheral blood, plasma and serum.

According to some embodiments, the cfDNA quantity is the total quantity of cfDNA in the fluid sample.

According to some embodiments, the cfDNA comprises cfDNA from the subject and cfDNA from the donor.

According to some embodiments, the method does not comprise isolating cfDNA only from the subject.

According to some embodiments, the predetermined threshold is the cfDNA quantity in a fluid sample from a healthy control subject or from a population of healthy control subjects.

According to some embodiments, the predetermined threshold is the cfDNA quantity in a fluid sample from a control subject that underwent an HCT and does not have GVHD.

According to some embodiments, the cfDNA is cfDNA originating from a specific tissue or cell type.

According to some embodiments, the specific tissue or cell type is selected from skin, lung, liver, intestine, B cells, T cells, CD8 positive T cells, eosinophils, monocytes, neutrophils and T regulatory cells (Tregs).

According to some embodiments, the specific tissue or cell type is selected from liver, B cells, T cells, CD8 positive T cells, eosinophils, monocytes, neutrophils and Tregs.

According to some embodiments, the cell type is an immune cell derived from a hematopoietic stem cell.

T According to some embodiments, the cfDNA is cfDNA originating from neutrophils, monocytes, eosinophils, T cell, CD8 cells, B cells, skin and liver.

According to some embodiments, the origin of the cfDNA was determined by
a. analyzing methylation of CpGs at an informative locus within the cfDNA, wherein the informative locus is uniquely methylated or unmethylated within the specific tissue or cell type so as to allow unique identification of the origin of the cfDNA based on the methylation status of the informative locus; or
b. chromatin immunoprecipitation sequencing (ChIP-Seq) and correlating binding of a cfDNA-associated protein with an informative locus within the cfDNA, wherein if the cfDNA-associated protein is indicative of active transcription the informative locus is uniquely actively transcribed within the specific tissue or cell type, and if the cfDNA-associated protein is indicative of silenced transcription the informative locus is uniquely silenced within the specific tissue or cell type, so as to allow unique identification of the origin of the cfDNA based on the protein associated with the informative locus.

According to some embodiments, the measurement of cfDNA was generated by performing methylation sensitive sequencing on the cfDNA to produce the nucleotide sequence of the cfDNA including the methylation status of each cytosine in the nucleotide sequence and assigning sequencing reads as originating from the specific tissue or cell type based on the reads' nucleotide sequence and methylation status.

According to another aspect, there is provided a method of classifying a sample as originating from a subject suffering from GVHD or a subject not suffering from GVHD, the method comprising:
a. receiving measurements of at least two parameters in a fluid sample, wherein the parameters are selected from the group consisting of: quantity of cfDNA originating from a specific tissue or cell type, liver enzyme level, white blood cell (WBC) count, and hemoglobin levels; and
b. applying a trained machine learning (ML) model to the at least two received parameters;
thereby classifying a sample.

According to another aspect, there is provided a method of classifying a sample as originating from a subject suffering from GVHD or a subject not suffering from GVHD, the method comprising:
a. receiving measurements of at least two parameters in a fluid sample, wherein the parameters comprise at least one clinical parameter and at least one cfDNA parameter, and wherein the clinical parameter is selected from the group consisting of: liver enzyme level, white blood cell (WBC) count, and hemoglobin level and the cfDNA parameter is selected from total cfDNA quantity and quantity of cfDNA from a specific tissue or cell type; and
b. applying a trained machine learning (ML) model to the at least two received parameters;
thereby classifying a sample.

According to some embodiments, the method is a method of diagnosing GVHD in a subject in need thereof, and the sample is from the subject.

According to some embodiments, the ML model was trained on a training set comprising the at least two parameters in subjects suffering and not suffering from GVHD and the ML model outputs a diagnosis of having GVHD or not having GVHD.

According to some embodiments, the liver enzymes are selected from aspartate transaminase (AST), alanine transaminase (ALT), and alkaline phosphatase (ALP). According to some embodiments, the liver enzymes are selected from aspartate transaminase (AST), alanine transaminase (ALT), alkaline phosphatase (ALP) and gamma-glutamyl transpeptidase (GGTp).

According to some embodiments, the at least two parameters are cfDNA from two different tissues, cell types or both.

According to some embodiments, the at least two parameters comprise liver enzyme levels, WBC count and hemoglobin levels.

According to some embodiments, the at least two parameters comprise cfDNA quantity from monocytes, cfDNA quantity from eosinophils, cfDNA quantity from T cells, cfDNA quantity from CD8 cells, cfDNA quantity from B cells, cfDNA quantity from skin and cfDNA quantity from liver.

According to some embodiments, the cfDNA parameter is selected from: total cfDNA quantity, cfDNA quantity from neutrophils, cfDNA quantity from monocytes, cfDNA quantity from eosinophils, cfDNA quantity from Tregs, cfDNA quantity from T cells, cfDNA quantity from CD8 cells, cfDNA quantity from B cells, cfDNA quantity from skin, cfDNA quantity from intestine, cfDNA quantity from lung and cfDNA quantity from liver.

According to some embodiments, the at least two parameters further comprise liver enzyme levels.

According to some embodiments, the liver enzyme level comprises AST levels and ALT levels.

According to some embodiments, the at least two parameters comprise ALT level and total cfDNA quantity.

According to some embodiments, the at least two parameters further comprise a third parameter selected from: cfDNA quantity from monocytes, cfDNA quantity from skin, GGTp level, cfDNA quantity from neutrophils and cfDNA quantity from eosinophils.

According to some embodiments, the at least two parameters comprise ALT level, total cfDNA quantity and cfDNA quantity from monocytes.

According to some embodiments, the method further comprises treating a subject diagnosed with GVHD with an anti-GVHD therapeutic agent.

According to some embodiments, the method further comprises tapering immunosuppression treatment to a subject that is not diagnosed with GVHD.

According to another aspect, there is provided a method comprising:
training a machine learning model to predict the presence of graft versus host disease (GVHD), on a training set, the method comprising:
   i. extracting cfDNA from a fluid sample; and
   ii. quantifying the amount of cfDNA from at least two tissues or cell types in the fluid sample;
wherein the training set is generated by labeling cfDNA quantities from at least two tissues or cell types as coming from a subject that suffers from GVHD or does not suffer from GVHD and compiling a plurality of cfDNA quantities from at least two tissues or cell types and their labels together to form the training set, wherein the plurality comprises labels of both subjects that suffer from GVHD and subjects that do not suffer from GVHD.

According to another aspect, there is provided a method comprising:
training a machine learning model to predict the presence of graft versus host disease (GVHD) in a subject, on a training set, the method comprising:
   i. extracting cfDNA from a fluid sample;
   ii. quantifying the amount of cfDNA in the fluid sample; and
   iii. determining at least one clinical parameter in the fluid sample;
wherein the training set is generated by labeling cfDNA quantities in samples and at least one clinical parameter as coming from a subject that suffers from GVHD or does not suffer from GVHD and compiling a plurality of cfDNA quantities, clinical parameters and their labels together to form the training set, wherein the plurality comprises labels of both subjects that suffer from GVHD and subjects that do not suffer from GVHD and wherein the clinical parameter is selected from the group consisting of: liver enzyme level, white blood cell count (WBC) and hemoglobin level.

According to some embodiments, the fluid sample is a plurality of fluid samples comprising fluid samples from both subjects that suffer from GVHD and subjects that do not suffer from GVHD.

According to some embodiments, the cfDNA quantities comprise total cfDNA quantities in the samples and the at least one clinical parameter comprises ALT level.

According to some embodiments, the cfDNA quantities further comprise cfDNA quantities from monocytes.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D: characterization of methylation biomarkers. Bar graphs of tissue-specificity of methylation markers for (1A) liver, (1B) intestine, (1C) lung epithelium, (1D) and skin.

FIG. 2: Diagram of the experimental design. One hundred and one plasma samples were collected from 101 individuals arriving for planned clinical follow-up, at the BMT day care unit. Upon each visit blood was drawn for regular blood tests and the patient underwent a full assessment by the treating physician which included cGVHD grading according to the 2014 National Institute of Health (NIH) criteria. Ninety-three samples were employed for modeling, excluding those with missing data. * Abbreviations: cf=cell free;. cGVHD=chronic Graft Versus Host Disease; AST—Aspartate transaminase; ALP—Alkaline phosphatase; GGTp—gamma glutamyl transpeptidase.

FIGS. 3A-3H: cfDNA levels correlate with clinical presence of cGVHD. (3A) Dot plot of level of total cfDNA in healthy volunteers, and allogeneic HCT patients with and without clinical signs of cGVHD. (3B-3E) Dot plots of tissue-specific cfDNA (genome equivalents per mL plasma) in healthy volunteers, and allogeneic HCT patients with and without clinical signs of cGVHD, using average signals from methylation markers of (3B) skin, (3C) gastrointestinal tract, (3D) liver and (3E) lung. Red line shows the median level. Each dot represents one plasma sample. Statistical analysis was performed using non-parametric two-tailed Mann-Whitney test. (3F-3H) Tissue-specific cfDNA in healthy volunteers and in allogeneic HCT patients in relation to clinical chronic GVHD score in (3F) skin, (3G) lung and (3H) liver. Each dot represents one plasma sample.

FIGS. 4A-4G: Immune-derived cfDNA levels correlate with clinical presence of cGVHD. Dot plots of level of levels of immune specific cfDNA in healthy volunteers, and allogeneic HCT patients with and without clinical signs of cGVHD, using median signals from methylation markers of (4A) neutrophils, (4B) monocytes, (4C) eosinophils, (4D) B cells, (4E) T cells, (4F) CD8+ T cells, and (4G) Tregs. Each dot represents one plasma sample. Statistical analysis was performed using non-parametric two-tailed Mann-Whitney test. Red line shows the median level.

FIGS. 5A-5D: Matrix of correlations between cfDNA and clinical parameters and specific organ cGVHD scoring among transplanted patients. (5A) Heatmaps of Spearman rank correlation coefficient and significance of correlations between cfDNA and clinical parameters. (*<0.05 **<0.01 ***<0.001). (5B) Spearman's rank correlation coefficient between cfDNA and clinical parameters is listed for each comparison. (5C) Heatmaps of Spearman rank correlation coefficient and significance of correlations between cfDNA and specific organ cGVHD scoring. (*<0.05 **<0.01 ***<0.001). (5D) Spearman's rank correlation coefficient between cfDNA and specific organ cGVHD scoring is listed for each comparison. cfDNAng/ml=Total cell free DNA levels in ng/ml.

FIGS. 6A-6B: Shapley analysis—constrained. (6A) Evaluation of the contribution of each feature (ALT, cfDNA ng/ml, cfMonocytes, cfSkin, GGTp, cfNeutrophils, cfEosinophils) to the model's prediction of cGVHD. Parameter space of the model was constrained to be non-negative for all coefficients, thus showing only features with a non-negligible coefficient. (6B) Bar graph of the SHAP value distributions for the features. The average absolute SHAP value for each individual feature is provided.

FIG. 7: Variant of metrics based on number of features. Visual representation of metrics (specificity, sensitivity, AUC, NPV, PPV) based on the addition of features according to their importance as determined by SHAP analysis. NPV-Negative Predictive Value, PPV-Positive Predictive Value.

FIGS. 8A-8B: Repeated 5-fold cross-validation results on the best feature set. (8A) Bar plot of metrics for solely clinical laboratory features (first column; ALT), solely cfDNA features (second column; cfDNAng/ml, cfMonocytes) and combined (third column; ALT, cfDNAng/ml, cfMonocytes) (8B) ROC curves for solely clinical laboratory features (red; ALT, AUC=0.65), solely cfDNA features (green; cfDNAng/ml, cfMonocytes; AUC=0.74) and combined (blue; ALT, cfDNAng/ml, cfMonocytes; AUC=0.81).

FIG. 9: Liver cfDNA levels correlate with liver enzyme levels. Liver enzyme levels: ALT, AST, ALP, GGTp—represented in units/L and TBIL levels in micromole/L. Each dot represents one plasma sample. ALT—Alanine transaminase, AST—Aspartate transaminase; ALP—Alkaline phosphatase; GGTp—gamma glutamyl transpeptidase; TBIL—Total bilirubin.

FIGS. 10A-10B: Shapley analysis—non constrained. Evaluation of the contribution of each feature to the model's prediction of cGVHD. (10A) Parameter space of the model was non constrained thus showing all 17 measured features. (10B) Bar graphs of the SHAP value distributions. The average absolute SHAP value is given for each individual feature (Total cell free DNA levels in ng/ml).

FIG. 11: Variation of Metrics based on Number of Features. Visual representation of metrics (specificity, sensitivity, AUC, NPV, PPV) based on the addition of all 17 features according to their importance as determined by SHAP analysis.

FIGS. 12A-12B: Repeated 5-fold cross validation results on the best feature set. (12A) Bar plot of metrics for solely clinical laboratory features (first column; ALT, GGTp, ALP), solely cfDNA features (second column; cfDNAng/ml, cfMonocytes, cfEosinophils, cfNeutrophils) and combined (thired column; ALT, GGTp, ALP, cfDNAng/ml, cfMonocytes, cfEosinophils, cfNeutrophils). (12B) ROC curves for solely clinical laboratory features (red; ALT, GGTp, ALP; AUC=0.68), solely cfDNA features (green; cfDNAng/ml, cfMonocytes, cfEosinophils, cfNeutrophils; AUC=0.74) and combined top 3 features (blue; ALT, cfDNAng/ml, cfMonocytes; AUC-0.80).

### DETAILED DESCRIPTION OF THE INVENTION

The present invention, in some embodiments, provides methods of diagnosing graft versus host disease (GVHD) comprising receiving a measurement of cell-free DNA (cfDNA) quantity in a sample, wherein a cfDNA quantity above a predetermined threshold indicates the subject suffers from GVHD. Methods of diagnosing GVHD comprising applying a machine learning model to at least two parameters from a sample are also provided.

The invention is based on the surprising finding that total cfDNA levels and cfDNA levels from specific organ/cell types can diagnose and prognose cGVHD. cfDNA has been shown to be elevate in many diseases, such as myocardial infection, stroke, sepsis and autoimmune diseases (e.g., Sjögren's syndrome and SLE). Further, there is a known correlation between cfDNA levels and severity of tissue damage. However, past studies (see Duque-Alfonso et al., “Cell-free DNA characteristics and chimerism analysis in patients after allogeneic cell transplantation”, Clin. Biochem., 2018, February:52:137-141) that looked at cfDNA levels in HCT recipients who developed GVHD and those that didn't did not find predictive value in total cfDNA levels. Indeed, in Dugue-Alfonso it was only reported that if cfDNA from donor cells was removed there was a correlation between recipient only cfDNA and development of GVHD. Such a measure does not have broad clinical value as it would require a diagnostic test specifically designed for every pair of donors and recipients in order to distinguish their cfDNA. In contrast, the instant invention discloses that total cfDNA, cfDNA from specific tissues and several combined cfDNA and patient data models can accurately predict chronic GVHD without the need to resort to patient tailored analyses. In particular, a machine learning model trained on total cfDNA quantities, and alanine transaminase (ALT) levels or total cfDNA levels, ALT levels and cfDNA quantities from monocytes was able to very accurately predict GVHD presence even before clinical manifestation.

By a first aspect, there is provided a method of diagnosing graft versus host disease (GVHD) in a subject, the method comprising receiving a measurement of cell-free DNA (cfDNA) in a sample from the subject, wherein cfDNA above a predetermined threshold indicates the subject has GVHD, thereby diagnosing GVHD.

By another aspect, there is provided a method of classifying a sample as originating from a subject suffering from GVHD or a subject not suffering from GVHD, the method comprising:
a. receiving measurements of at least two parameters in a sample; and
b. applying a trained machine learning (ML) model to the at least two received parameters;
thereby diagnosing GVHD in a subject.

By another aspect, there is provided a method of diagnosing GVHD in a subject, the method comprising:
a. receiving measurements of at least two parameters in a sample from the subject; and
b. applying a trained machine learning (ML) model to the at least two received parameters;
thereby diagnosing GVHD in a subject.

By another aspect, there is provided a method comprising:
training a machine learning (ML) model to predict the presence of GVHD, on a training set, the method comprising:
   i. extracting cfDNA from a sample; and
   ii. quantifying the cfDNA from at least two tissues or cell types in the sample.

By another aspect, there is provided a method comprising: training a machine learning (ML) model to predict the presence of GVHD, o a training set, the method comprising receiving cfDNA quantities from at least two tissues or cell types measured in a fluid sample.

By another aspect, there is provided a method comprising:
training a machine learning (ML) model to predict the presence of GVHD, on a training set, the method comprising:
   i. extracting cfDNA from a sample; and
   ii. quantifying the cfDNA in the sample.

By another aspect, there is provided a method comprising:
training a machine learning (ML) model to predict the presence of GVHD, on a training set, the method comprising:
   i. receiving a cfDNA quantity measured in a fluid sample; and
   ii. receiving a clinical parameter measured in the fluid sample.

Graft-versus-host-disease (GVHD) is a life-threatening, complication that can arise following allogeneic hematopoietic cell transplantation. GVHD is the leading cause of post-transplantation morbidity and non-relapse mortality in hematopoietic stem cell transplants (HSCTs) and poses the greatest threat to transplantation success. GVHD can be classified as acute or chronic. Acute GVHD refers to GVHD (maculopapular rash, nausea, vomiting, anorexia, profuse diarrhea, ileus, or cholestatic hepatitis) occurring within 100 days after transplantation or Donor Lymphocyte Infusion (DLI). Chronic GVHD is distinctive from acute GVHD and is not simply an evolution of acute GVHD. Chronic GVHD is a syndrome of variable clinical features resembling autoimmune and other immunologic disorders such as scleroderma. The pathophysiology of the chronic GVHD syndrome may involve inflammation, cell-mediated immunity, humoral immunity, and fibrosis. Clinical manifestations nearly always present during the first year after transplantation, but some cases develop many years after HCT. Manifestations of chronic GVHD may be restricted to a single organ or site or may be widespread, with profound impact on quality of life. In some embodiments, GVHD is chronic GVHD. In some embodiments, GVHD is acute GVHD. In some embodiments, GVHD is chronic and/or acute GVHD.

In some embodiments, the method is an in vitro method. In some embodiments, the method is an ex vivo method. In some embodiments, the method is a diagnostic method. In some embodiments, the method is a method of detecting GVHD. In some embodiments, the method is a method of predicting GVHD. In some embodiments, the method is a method of predicting the presence of GVHD. In some embodiments, the method is a method assessing the risk. In some embodiments, the risk is risk of developing GVHD. In some embodiments, risk is risk of having GVHD.

In some embodiments, the subject is a mammal. In some embodiments, the subject is a human. In some embodiments, the subject is in need of a method of the invention. In some embodiments, the subject is in need of treatment. In some embodiments, the subject has received a hematopoietic stem cell transplant (HCT). In some embodiments, the subject underwent an HCT. In some embodiments, the subject has undergone an HCT. In some embodiments, the HCT is allogeneic stem cell transplant. In some embodiments, the HCT is reduced-intensity allogeneic stem cell transplant. In some embodiments, the HCT is from a donor. In some embodiments, the donor is the person that provided the stem cells for transplant to the subject. In some embodiments, the subject underwent the HCT within the last 100 days. In some embodiments, the subject underwent the HCT more than 100 days ago. In some embodiments, the subject underwent the HCT more than 100 days before the sample was taken. In some embodiments, the subject has not been diagnosed with GVHD. In some embodiments, the subject has not been diagnosed with acute GVHD. In some embodiments, the subject has not been diagnosed with chronic GVHD. In some embodiments, the subject has been diagnosed with acute GVHD. In some embodiments, the subject is at risk of developing GVHD.

In some embodiments, the sample is a fluid sample. In some embodiments, the fluid is a bodily fluid. In some embodiments, the bodily fluid is selected from at least one of: blood, serum, plasma, gastric fluid, intestinal fluid, saliva, bile, tumor fluid, breast milk, urine, interstitial fluid, cerebral spinal fluid and stool. In some embodiments, the bodily fluid is selected from blood, serum and plasma. In some embodiments, the bodily fluid is blood. In some embodiments, the blood is peripheral blood. In some embodiments, the sample is a sample that comprises cfDNA. In some embodiments, the sample is depleted of cells. In some embodiments, the sample obtained from the subject comprises cells and the cells are removed. In some embodiments, cfDNA is isolated from the sample. In some embodiments, the sample is a sample of isolated cfDNA. In some embodiments, the sample is isolated cfDNA from a sample obtained from the subject. In some embodiments, the sample is an isolated sample. In some embodiments, the sample is an enriched sample. In some embodiments, enriched is enriched for cfDNA. In some embodiments, the sample is a purified sample.

In some embodiments, the sample is from the subject. In some embodiments, the sample is from a subject. In some embodiments, the method is a method of diagnosing a subject and the sample is from the subject. In some embodiments, classifying a sample comprises diagnosing the subject. In some embodiments, classifying a sample as originating from a subject suffering from GVHD comprises diagnosing the subject as having GVHD. In some embodiments, classifying a sample as originating from a subject that does not suffer from GVHD comprises diagnosing the subject as not having GVHD.

In some embodiments, the method further comprises obtaining the sample from the subject. In some embodiments, the method further comprises extracting the sample from the subject. In some embodiments, the method further comprises receiving the sample. In some embodiments, the method further comprises depleting the sample of cells. In some embodiments, cells are intact cells. In some embodiments, cells are cellular debris. In some embodiments, the method further comprises isolating the cfDNA. In some embodiments, the method further comprises enriching for cfDNA. In some embodiments, the method further comprises purifying the cfDNA. In some embodiments, the method further comprises measuring the cfDNA. In some embodiments, the isolating/enriching/purifying is isolating/enriching/purifying cfDNA from the donor. In some embodiments, the isolating/enriching/purifying is not isolating/enriching/purifying cfDNA from the subject. In some embodiments, the method does not comprise isolating or purifying cfDNA only from the subject. In some embodiments, the method does not comprise measuring cfDNA only from the subject.

As used herein the terms “separating”, “excluding” or “isolating” is intended to mean that the material has been completely, substantially or partially separated, isolated, excluded or purified from other components, e.g., cells, or cell fragments including but not limited to membranes, proteins or nucleic acid molecules. As used herein, the term “isolated cfDNA” refers to cfDNA that is essentially free from contaminating cellular components, such as carbohydrate, lipid, proteins or other nucleic acid molecules such as genomic DNA or mRNA. In some embodiments, an isolated cfDNA is a purified cfDNA. In some embodiments, an isolated and/or purified cfDNA is at least 80% pure, at least 85% pure, at least 90% pure, at least 95% pure, at least 97% pure, at least 99% pure or 100% pure. Each possibility represents a separate embodiment of the invention. In some embodiments, an isolated and/or purified cfDNA is at least 80% pure. In some embodiments, an isolated and/or purified cfDNA is at least 90% pure.

As used herein, the term “cfDNA” refers to non-encapsulated DNA that is found in an organism outside of a cell. cfDNA is generally degraded DNA fragments of a size of about 50-220 nucleotides that is released for dying cells. In some embodiments, cfDNA is a small DNA molecule. In some embodiments, cfDNA is from apoptotic cells. In some embodiments, cfDNA is from necrotic cells. In some embodiments, cfDNA is cfDNA from cells killed by the immune system. In some embodiments, cfDNA is from cells of the immune system. In some embodiments, cfDNA is not cell-free fetal DNA (cfDNA). In some embodiments, cfDNA is not circulating tumor DNA (ctDNA). In some embodiments, cfDNA is not cell free mitochondrial DNA. In some embodiments, cfDNA is double stranded DNA. In some embodiments, cfDNA is single stranded DNA. In some embodiments, cfDNA is cell free nucleosomes. In some embodiments, cfDNA comprises DNA associated proteins. In some embodiments, the DNA associated proteins are histones. In some embodiments, cfDNA is methylated DNA. In some embodiments, cfDNA is unmethylated DNA. In some embodiments, cfDNA is cfDNA from the subject. In some embodiments, cfDNA is not cfDNA only from the subject. In some embodiments, cfDNA is cfDNA from the donor. In some embodiments, cfDNA is cfDNA from the subject and the donor. In some embodiments, cfDNA is cfDNA from a particular tissue or cell type.

In some embodiments, the sample is enriched for small DNA molecules. In some embodiments, small is smaller than 1000, 950, 900, 850, 800, 750, 700, 650, 600, 550, 500, 450, 400, 350, 300, 290, 280, 275, 270, 260, 250, 240, 230, 225, 220, 215, 210, 205, 200, 195, 190, 185, 180, 175, 170, 169, 168, 167, 166, 165, 160, 155 or 150 nucleotides. Each possibility represents a separate embodiment of the invention. In some embodiments, small is less than 500 nucleotides. In some embodiments, small is less than 220 nucleotides. In some embodiments, small is less than 200 nucleotides. In some embodiments, small is less than 169 nucleotides. In some embodiments, small is bigger than 1, 2, 3, 4, 5, 6, 7, 8, 9, 10, 12, 15, 17, 20, 25, 30, 35, 40, 45, or 50 nucleotides. Each possibility represents a separate embodiment of the invention.

In some embodiments, the method comprises a size selection step. In some embodiments, the sample is size selected. In some embodiments, size selection is selection for small DNAs. In some embodiments, the size selection is SPRI bead size selection. In some embodiments, SPRI selection is SPRI bead size exclusion. SPRI beads are well known in the art and can be used to isolate DNA. By altering the concentration of SPRI beads one can alter the size of DNA that tends to bind. Increased numbers of beads lead to binding of smaller DNAs and fewer beads lead to binding of larger DNAs. In some embodiments, the concentration of SPRI beads is increased. In some embodiments, increased is as compared to a standard protocol. In some embodiments, the ratio of bead to sample is increased. In some embodiments, the ratio of bead to sample is at least 1:1, 1.1:1, 1.2:1, 1.25:1, 1.3:1, 1.4:1, 1.5:1, 1.6:1, 1.7:1, 1.75:1, 1.8:1, 1.9:1 or 2:1. Each possibility represents a separate embodiment of the invention. In some embodiments, the ratio of bead to sample is at least 1.8:1. In some embodiments, the ratio of bead to sample is about 1.8:1. In some embodiments, the ratio of bead to sample is at most 1.8:1, 1.9:1, 2:1, 2.1:1, 2.2:1, 2.25:1, 2.3:1, 2.4:1, 2.5:1, 2.6:1, 2.7:1, 2.75:1, 2.8:1, 2.9:1, 3:1, 3.5:1, 4:1, 4.5:1, 5:1. Each possibility represents a separate embodiment of the invention.

In some embodiments, measuring cfDNA comprises measuring cfDNA quantity. In some embodiments, measuring cfDNA comprises determining the source of the cfDNA. In some embodiments, measuring cfDNA does not comprises determining the source of the cfDNA. In some embodiments, measuring cfDNA does not comprises determining if the cfDNA is from cells from the donor or the subject. In some embodiments, measuring cfDNA comprises identifying the source of the cfDNA. In some embodiments, the source of the cfDNA is the tissue, cell type or cell of origin of the cfDNA. In some embodiments, the source of the cfDNA is the tissue or cell type of origin of the cfDNA. In some embodiments, the source of the cfDNA is the cell that died and released the cfDNA. In some embodiments, measuring cfDNA quantity comprises measuring the quantity of cfDNA from a particular source. In some embodiments, measuring cfDNA quantity comprises measuring the quantity of cfDNA from a plurality of sources. In some embodiments, measuring cfDNA quantity comprises measuring the quantity of cfDNA from the subject. In some embodiments, measuring cfDNA quantity comprises measuring the quantity of cfDNA from the donor. In some embodiments, measuring cfDNA quantity comprises measuring the quantity of cfDNA from the donor and the subject. In some embodiments, cfDNA quantity is the total quantity of cfDNA in the sample.

In some embodiments, identification of DNA methylation or histone modification at an informative genetic locus indicates the tissue of origin of the DNA. In some embodiments, identification of DNA methylation or histone modification at an informative genetic locus indicates the cell type of origin of the DNA. In some embodiments, identification of DNA methylation or histone modification at an informative genetic locus indicates the DNA originated from a particular tissue. In some embodiments, identification of DNA methylation or histone modification at an informative genetic locus indicates the DNA originated from a particular cell type. In some embodiments, identification of DNA unmethylation or lack of a modified histone at an informative genetic locus indicates the tissue of origin of the DNA. In some embodiments, identification of DNA unmethylation or lack of a modified histone at an informative genetic locus indicates the cell type of origin of the DNA. In some embodiments, identification of DNA unmethylation or lack of a modified histone at an informative genetic locus indicates the DNA originated from a particular tissue. In some embodiments, identification of DNA unmethylation or lack of a modified histone at an informative genetic locus indicates the DNA originated from specific cell type.

In some embodiments, the origin of the cfDNA was determined by analyzing methylation of CpGs at an informative locus within the cfDNA. In some embodiments, methylation is DNA methylation. In some embodiments, CpGs are CpG dinucleotides. In some embodiments, the genetic locus is selected from a genetic locus provided in any one of International Patent Applications WO2019012542, WO2019012543, WO2019175876 and WO2020212992. In some embodiments, the informative locus is uniquely methylated within the specific tissue or cell type. In some embodiments, the informative locus is uniquely unmethylated within the specific tissue or cell type. In some embodiments, unique is within the specific tissue or cell type and not in other tissues or cell types. In some embodiments, unique methylation/unmethylation allows identification of the origin of the cfDNA. In some embodiments, the identification is unique identification. In some embodiments, the identification is based on the methylation status of the informative locus. In some embodiments, methylation status is methylation or unmethylation. In some embodiments, the genetic locus is selected from a genetic locus provided herein. In some embodiments, the genetic locus is selected from a genetic locus identified by the primers provided in Table 1.

In some embodiments, the measurement of cfDNA comprises measurement of cfDNA methylation. In some embodiments, the measurement of cfDNA methylation comprises performing methylation sensitive sequencing. In some embodiments, the sequencing is next generation sequencing. In some embodiments, the sequencing produces a nucleotide sequence of the cfDNA. In some embodiments, the nucleotide sequence includes methylation status of a cytosine in the sequence. In some embodiments, the nucleotide sequence includes the methylation status of each cytosine in the sequence. In some embodiments, the nucleotide sequence includes the methylation status of all cytosines in the sequence. In some embodiments, the measurement comprises assigning sequencing reads to a specific tissue or cell type of origin. In some embodiments, the measurement comprises quantifying the sequencing reads that originate from a specific tissue or cell type. In some embodiments, the measurement is the quantity of reads. In some embodiments, the measurement is the relative number of reads. In some embodiments, the measurement is the genome equivalent number of reads. In some embodiments, the assigning is based on the combination of the nucleotide sequence and methylation status.

In some embodiments, the measurement of cfDNA comprises measurement of cfDNA associated proteins. In some embodiments, the measurement of cfDNA associated proteins comprises performing chromatin immunoprecipitation sequencing (ChIP-Seq). In some embodiments, the sequencing is next generation sequencing. In some embodiments, the sequencing produces a nucleotide sequence of the cfDNA associated with specific proteins. In some embodiments, the specific proteins are DNA binding proteins. In some embodiments, the specific proteins are selected from histones, histone variants, post-translationally modified histones, high mobility group (HMG) proteins and transcription factors. In some embodiments, the specific proteins are histones. In some embodiments, the histone is a histone variant. In some embodiments, the histone is a histone comprising a specific post-translational modification. According to some embodiments, the DNA-associated protein is selected from a histone, a high-mobility group (HMG) protein and a member of the transcriptional machinery. According to some embodiments, the histone is a histone variant and/or a modified histone. Examples of modified histones include, but are not limited to, Histone 3 monomethylated lysine 4 (H3K4me1), Histone 3 demethylated lysine 4 (H3K4me2), Histone 3 trimethylated lysine 36 (H3K36me3) and Histone 3 trimethylated lysine 4 (H3K4me3). In some embodiments, an antibody against the cfDNA associated protein is added to the sample and cfDNA associated with that protein is isolated. In some embodiments, the measurement comprises assigning sequencing reads to a specific tissue or cell type of origin. In some embodiments, the measurement comprises quantifying the sequencing reads that originate from a specific tissue or cell type. In some embodiments, the measurement is the quantity of reads. In some embodiments, the measurement is the relative number of reads. In some embodiments, the measurement is the genome equivalent number of reads. In some embodiments, the assigning is based on the combination of the nucleotide sequence and the protein associated with the cfDNA containing the sequence.

According to some embodiments, association of the DNA-associated protein with the genomic location is indicative of active transcription and the genomic location is within a tissue or cell type specific gene or enhancer element. According to some embodiments, association of the DNA-associated protein with the genomic location is indicative of silenced transcription and the genomic location is within a repressor element, or a gene silenced in the tissue or cell type.

Methods of measuring cfDNA are well known in the art and any such method may be employed. Measuring cfDNA quantity may be performed for example with a nanopore, a sequencer, a spectrophotometer (e.g., a NanoDrop), a fluorometer, and electrophoresis to name but a few. In some embodiments, the sequencer is a next-generation sequencer. In some embodiments, the measuring comprises sequencing the cfDNA. Methods of measuring or identifying the tissue or cell type of origin of cfDNA are also well known in the art. They include sequence analysis, DNA methylation analysis, analysis of DNA associated proteins (e.g., histone modification or variant analysis) and combinations of sequence analysis and epigenetic analysis (e.g., DNA methylation/histone modification). Methods of cfDNA source analysis and lists of informative loci can also be found in, for example, J. Moss, et al., “Comprehensive human cell-type methylation atlas reveals origins of circulating cell-free DNA in health and disease” Nat. Commun. 9, 5068 (2018); F. Mouliere, et al., “Enhanced detection of circulating tumor DNA by fragment size analysis” Sci. Transl. Med. 10 (2018); and International Patent Applications WO2019012542, WO2019012543, WO2019175876 and WO2020212992 herein incorporated by reference in their entirety.

In some embodiments, the cfDNA is total cfDNA. In some embodiments, total cfDNA comprises cfDNA from the donor and the subject. In some embodiments, the cfDNA is cfDNA originating from the donor. In some embodiments, the cfDNA is cfDNA originating from a specific tissue or cell type of the subject. In some embodiments, the cfDNA is cfDNA originating from a specific tissue or cell type. In some embodiments, the specific cell type is a hematopoietic cell. In some embodiments, the specific cell type is an immune cell. In some embodiments, the specific cell type is a cell derived from a hematopoietic stem cell. In some embodiments, the specific tissue or cell type is selected from skin, lung, liver, colon, B cells, T cells, CD8 positive T cells, eosinophils, monocytes, neutrophils and T regulatory cells (Tregs). In some embodiments, the specific tissue or cell type is selected from skin, lung, liver, intestine, B cells, T cells, CD8 positive T cells, eosinophils, monocytes, neutrophils and T regulatory cells (Tregs). In some embodiments, the specific tissue or cell type is selected from liver, B cells, T cells, CD8 positive T cells, eosinophils, monocytes, neutrophils and Tregs. In some embodiments, the specific tissue is skin. In some embodiments, the specific tissue is lung. In some embodiments, the specific tissue is liver. In some embodiments, the specific tissue is colon. In some embodiments, colon is intestine. In some embodiments, intestine is colon. In some embodiments, colon is the gastrointestinal (GI) tract. In some embodiments, intestine is the GI tract. In some embodiments, the specific cell type is selected from B cells, T cells, CD8 positive T cells, eosinophils, monocytes, neutrophils and T regulatory cells (Tregs). In some embodiments, the specific cell type is eosinophils. In some embodiments, the specific cell type is monocytes. In some embodiments, the specific cell type is neutrophils. In some embodiments, the specific cell type is B cells. In some embodiments, the specific cell type is T cells. In some embodiments, the specific cell type is CD8 positive cells. In some embodiments, CD8 positive cells are CD8 positive T cells. In some embodiments, CD8 positive cells are all CD8 positive cells. In some embodiments, the specific cell type is Tregs.

In some embodiments, the cfDNA is cfDNA originating from a plurality of specific tissues and/or cell types. In some embodiments, a plurality is at least 2, 3, 4, 5, 6, 7, 8, 9, 10 or 11 tissues and/or cell types. Each possibility represents a separate embodiment of the invention. In some embodiments, a plurality is at least 3 tissues and/or cell types. In some embodiments, a plurality is at least 5 tissues and/or cell types. In some embodiments, a plurality is at least 7 tissues and/or cell types. In some embodiments, the cfDNA is cfDNA originating from monocytes, eosinophils, T cell, CD8 cells, B cells, skin and liver.

In some embodiments, the predetermined threshold is the cfDNA measurement in a sample from a healthy control. In some embodiments, the predetermined threshold is the average of cfDNA measurements in a group of samples from a population of healthy controls. In some embodiments, the cfDNA measurement is the cfDNA quantity. In some embodiments, a healthy control is a subject without GVHD. In some embodiments, a healthy control is a subject that has not underwent an HCT. In some embodiments, a healthy control is a subject that underwent an HCT and does not have GVHD. In some embodiments, a healthy control is a subject that underwent an HCT and did not develop GVHD.

As used herein, the term “parameter” refers to any measurable characteristic of the sample. In some embodiments, measurements of at least two parameters are received. In some embodiments, at least two is at least 2, 3, 4, 5, 6, 7, 8, 9, or 10 parameters. Each possibility represents a separate embodiment of the invention. In some embodiments, at least 2 is at least 3. In some embodiments, at least 2 is at least 5. In some embodiments, at least 2 is at least 6. In some embodiments, at least 2 is at least 7. In some embodiments, at least 2 is at least 8. In some embodiments, at least 2 is at least 9.

In some embodiments, the parameter is a cfDNA parameter. In some embodiments, a parameter is a quantity of cfDNA. In some embodiments, the quantity of cfDNA is the total quantity of cfDNA. In some embodiments, cfDNA is cfDNA in the sample. In some embodiments, the quantity of cfDNA is quantity of cfDNA form a specific tissue or cell type. In some embodiments, a specific tissue or cell type is a plurality of specific tissues and/or cell types. In some embodiments, the plurality is selected from neutrophils, monocytes, eosinophils, T cells, CD8 cells, B cells, Tregs, skin, lung, intestine and liver. In some embodiments, the plurality is selected from neutrophils, monocytes, eosinophils, T cells, CD8 cells, B cells, skin and liver. In some embodiments, the plurality is selected from monocytes, eosinophils, T cells, CD8 cells, B cells, skin and liver. In some embodiments, the plurality is selected from monocytes, eosinophils, T cells, CD8 cells, and B cells. In some embodiments, the at least two parameters comprise cfDNA from monocytes quantity, cfDNA from eosinophils quantity, cfDNA from T cell quantity, cfDNA from CD8 cells quantity, cfDNA from B cells quantity, cfDNA from skin quantity and cfDNA from liver quantity.

In some embodiments, the cfDNA quantity is a cfDNA parameter. In some embodiments, a cfDNA parameter is a cfDNA amount. In some embodiments, an amount is a quantity. In some embodiments, the cfDNA parameter is selected from: total cfDNA quantity, cfDNA quantity from neutrophils, cfDNA quantity from monocytes, cfDNA quantity from eosinophils, cfDNA from Treg cells, cfDNA quantity from T cells, cfDNA quantity from CD8 cells, cfDNA quantity from B cells, cfDNA quantity from skin, cfDNA quantity from intestine, cfDNA quantity from lung and cfDNA quantity from liver. In some embodiments, the cfDNA parameter is total cfDNA quantity and cfDNA quantity from at least one specific tissue and/or cell type. In some embodiments, the cfDNA parameter is total cfDNA quantity and at least one of: cfDNA quantity from neutrophils, cfDNA quantity from monocytes, cfDNA quantity from eosinophils, cfDNA from Treg cells, cfDNA quantity from T cells, cfDNA quantity from CD8 cells, cfDNA quantity from B cells, cfDNA quantity from skin, cfDNA quantity from intestine, cfDNA quantity from lung and cfDNA quantity from liver. In some embodiments, the cfDNA parameter is total cfDNA quantity and at least one of: cfDNA quantity from monocytes, cfDNA quantity from skin, cfDNA quantity from neutrophils and cfDNA quantity from eosinophils. In some embodiments, the cfDNA parameter is total cfDNA quantity and cfDNA quantity from monocytes.

In some embodiments, the parameter is a clinical parameter. In some embodiments, a clinical parameter is a non-cfDNA parameter. In some embodiments, a clinical parameter is a blood parameter. In some embodiments, a blood parameter is a parameter that can be measured from a blood sample but does not comprise sequencing of cfDNA. In some embodiments, the parameter is a blood parameter. In some embodiments, the parameter is a blood cell count. In some embodiments, the blood cell count is white blood cells count (WBC). In some embodiments, the blood cell count is red blood cell count. In some embodiments, the parameter is a blood protein level. In some embodiments, the blood protein is hemoglobin.

In some embodiments, the blood protein is a liver enzyme. In some embodiments, the parameter is a liver enzyme. In some embodiments, the liver enzyme is a secreted liver enzyme. In some embodiments, the liver enzyme is a circulating liver enzyme. In some embodiments, the liver enzyme is a plurality of liver enzymes. In some embodiments, the liver enzyme is selected from aspartate transaminase (AST), alanine transaminase (ALT), alkaline phosphatase (ALP) and gamma-glutamyl transpeptidase (GGTp). In some embodiments, the liver enzyme is selected from AST and ALT. In some embodiments, the liver enzyme is ALT. In some embodiments, the liver enzyme is AST. In some embodiments, the liver enzyme is ALP. In some embodiments, the liver enzyme is GGTp. In some embodiments, the at least two parameters comprise liver enzyme levels, WBC count and hemoglobin levels. In some embodiments, the at least two parameters comprise liver enzyme levels and WBC count. In some embodiments, the at least two parameters comprise AST and ALT levels, WBC count and hemoglobin levels. In some embodiments, the at least two parameters comprise AST and ALT levels and WBC count.

In some embodiments, the at least two parameters comprise a cfDNA parameter and a clinical parameter. In some embodiments, the at least two parameters comprise a liver enzyme level and a cfDNA parameter. In some embodiments, the at least two parameters comprise a liver enzyme level and total cfDNA quantity. In some embodiments, the at least two parameters are selected from: ALT levels, total cfDNA quantity, cfDNA quantity from monocytes, cfDNA quantity from skin, GGTp levels, cfDNA quantity from neutrophils, and cfDNA quantity from eosinophils. In some embodiments, the at least two parameters comprise ALT level and total cfDNA quantity. In some embodiments, the clinical parameter is ALT level and the cfDNA parameter is total cfDNA quantity. In some embodiments, the at parameters comprise ALT level and total cfDNA quantity and further comprise at least one of: cfDNA quantity from monocytes, cfDNA quantity from skin, GGTp levels, cfDNA quantity from neutrophils, and cfDNA quantity from eosinophils. In some embodiment, the at least two parameters further comprise at least a third parameter selected from: cfDNA quantity from monocytes, cfDNA quantity from skin, GGTp level, cfDNA quantity from neutrophils and cfDNA quantity from eosinophils. In some embodiments, the at least two parameters are at least three parameters and comprise ALT level, total cfDNA quantity and cfDNA quantity from monocytes.

In some embodiments, the at least two parameters comprise cfDNA from monocytes quantity, cfDNA from eosinophils quantity, cfDNA from T cell quantity, cfDNA from CD8 cells quantity, cfDNA from B cells quantity, cfDNA from skin quantity, cfDNA from liver quantity and liver enzyme levels. In some embodiments, the at least two parameters comprise cfDNA from monocytes quantity, cfDNA from eosinophils quantity, cfDNA from T cell quantity, cfDNA from CD8 cells quantity, cfDNA from B cells quantity, cfDNA from skin quantity, cfDNA from liver quantity and AST and ALT levels.

In some embodiments, the ML model was trained on a training set. In some embodiments, the training set comprises the at least two parameters. In some embodiments, the training set comprises measurements of the at least two parameters. In some embodiments, the training set comprises at least one cfDNA parameter and at least one clinical parameter. In some embodiments, the parameters in the training set are from subjects suffering from GVHD. In some embodiments, the parameters in the training set are from healthy subjects. In some embodiments, healthy subjects are subjects not suffering from GVHD. In some embodiments, the parameters in the training set are from subjects suffering and not suffering from GVHD. In some embodiments, the parameters in the training set are from subjects suffering GVHD and healthy subjects. In some embodiments, the parameters are labeled. In some embodiments, the label indicates the source of the parameter. In some embodiments, the source is the subject that provided the sample from which the parameter was determined. In some embodiments, the label is GVHD or not GVHD. In some embodiments, the label is from a subject with GVHD or from a subject without GVHD. In some embodiments, the label is from a subject suffering from GVHD or from a healthy subject.

In some embodiments, the machine learning model is trained to predict GVHD presence. In some embodiments, the machine learning model is trained to detect GVHD presence. In some embodiments, the machine learning model is trained to diagnose GVHD. In some embodiments, the ML model outputs a diagnosis. In some embodiments, the ML model outputs a prediction. In some embodiments, diagnosis or prediction is having GVHD or not having GVHD. In some embodiments, the ML model outputs a GVHD probability. In some embodiments, the output is binary and consists of GVHD or no GVHD. In some embodiments, the output is quantitative and provides a probability score.

Machine learning models are well known in the art and any such model may be used. Models include, but are not limited to artificial neural networks, support vector machines (SVM) classifier and a k-nearest neighbor (k-NN) classifier. In some embodiments, the machine learning model is a classification model. In some embodiments, the machine learning model is a classifier. In some embodiments, the machine learning model is an SVM classifier. In some embodiments, the machine learning model is a k-NN classifier. In some embodiments, the machine learning model is selected from an SVM classifier and a k-NN classifier. In some embodiments, the algorithm is a boosting algorithm. In some embodiments, the ML model employs the algorithm. In some embodiments, the ML model is the algorithm. In some embodiments, the algorithm is a random forest algorithm. In some embodiments, the machine learning model implements a machine learning algorithm. In some embodiments, the machine learning model is a supervised model. In some embodiments, supervised is self-supervised.

In some embodiments, the method further comprises an inference stage. In some embodiments, the inference stage comprises applying the trained ML model. In some embodiments, the trained ML model is applied to the parameters. In some embodiments, the parameters are the received parameters. In some embodiments, the inference stage comprises diagnosing/predicting GVHD. In some embodiments, the inference stage is for diagnosing/predicting GVHD.

In some embodiments, the training set is generated. In some embodiments, the generating comprises compiling parameters. In some embodiments, compiling is combining. In some embodiments, the method comprises producing the training set. In some embodiments, the method comprises obtaining a sample. In some embodiments, the method comprises extracting cfDNA from the sample. In some embodiments, the method comprises quantifying the cfDNA. In some embodiments, the quantifying is quantifying the amount of cfDNA. In some embodiments, cfDNA from a plurality of tissues and/or cell types is quantified. In some embodiments, the training set is generated by labeling the cfDNA quantities. In some embodiments, the label is the origin of the cfDNA. In some embodiments, the origin is from a subject that suffers from GVHD or a subject that does not suffer from GVHD. In some embodiments, the origin is from a subject that suffers from GVHD or a healthy subject. In some embodiments, the healthy subject is a subject that has had a HSCT. In some embodiments, the generating comprises compiling a plurality of cfDNA quantities. In some embodiments, the cfDNA quantities are from a plurality of tissues and/or cell types. In some embodiments, the generating comprises compiling the labels. In some embodiments, the compiling is compiling the cfDNA quantities and their labels. In some embodiments, the plurality comprises labels from both subject that suffer from GVHD and subjects that do not suffer from GVHD. In some embodiments, the plurality comprises labels from both subject that suffer from GVHD and healthy subjects. In some embodiments, the training set compiles parameters and labels from subjects that suffer from GVHD and subjects that do not suffer from GVHD. In some embodiments, the training set compiles parameters and labels from subjects that suffer from GVHD and healthy subjects.

In some embodiments, the method further comprises treating a subject diagnosed with GVHD. In some embodiments, the treating is with an anti-GVHD therapy. In some embodiments, the anti-GVHD therapy comprises an anti-GVHD therapeutic agent. In some embodiments, the treating comprises administering the anti-GVHD therapy. In some embodiments, the therapeutic agent is an immunosuppressant. In some embodiments, the therapeutic agent is an anti-lymphocyte agent. In some embodiments, the lymphocyte is a T cell. In some embodiments, the lymphocyte is a natural killer cell. In some embodiments, the therapeutic agent is a steroid. In some embodiments, the steroid is a corticosteroid. In some embodiments, the steroid is prednisone. In some embodiments, the steroid is methylprednisolone. In some embodiments, the therapeutic agent is an anti-tumor necrosis factor alpha (TNFa) agent. In some embodiments, the anti-TNFa agent is an anti-TNFa antibody. In some embodiments, the antibody is a blocking antibody. In some embodiments, the subject is already receiving a steroid and the treating comprises administering an increased dose of the steroid. In some embodiments, the subject is already receiving a steroid and the treating comprises administering a different steroid. In some embodiments, the different steroid is a stronger steroid. In some embodiments, stronger comprises a stronger immunosuppressing effect. Anti-GVHD therapies are well known in the art and any such therapy may be used. Examples of anti-GVHD therapies include, for example, steroids, cannabidiol (CBD), antithymocyte globulin, cyclosporin, mycophenolate mofetil, mesenchymal stem cell transplant, infliximab, alemtuzumab, daclizumab, extracorporeal photopheresis, sirolimus, and pentostatin.

In some embodiments, the method further comprises not treating a subject not diagnosed with GVHD. In some embodiments, the method further comprises reducing immunosuppression of a subject not diagnosed with GVHD. In some embodiments, reducing is tapering. In some embodiments, immunosuppression comprises administering an immunosuppressant. In some embodiments, the immunosuppressant is steroids. In some embodiments, reducing is ceasing. In some embodiments, reducing is stopping.

By another aspect, there is provided a system comprising: at least one hardware processor; and a non-transitory computer-readable storage medium having stored thereon program code, the program code executable by the at least one hardware processor to perform a method of the invention.

By another aspect, there is provided a computer program product comprising a non-transitory computer-readable storage medium having program code embodied therewith, the program code executable by at least one hardware processor to perform a method of the invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Methylation analysis: cfDNA was prepared and its concentration measured (in nanograms per milliliter plasma), then it was treated with bisulfite to expose the status of methylation and multiplex PCR (Neiman et al., “Multiplexing DNA methylation markers to detect circulating cell-free DNA derived from human pancreatic B cells, JCI Insight, 2020; 5(14), the contents of which are hereby incorporated by reference in its entirety) was performed to amplify marker loci. PCR products were sequenced on a NextSeq machine, and the fraction of molecules carrying a tissue-specific pattern of methylation was determined. This information, averaged over the markers for each tissue, was used to assess the relative contribution of each tissue/cell type to cfDNA. In addition, by multiplying the proportion of cfDNA from each tissue/cell type by the total concentration of cfDNA in a sample, the absolute concentration of cfDNA from each tissue in plasma was calculated and expressed in genome equivalents per milliliter plasma. Primer sequences of all markers are provided in Table 1.

TABLE 1
Primer sequences

| | Marker | Primer1 (SEQ ID NO:) | Primer2 (SEQ ID NO:) |
|---|---|---|---|
| B3GNTL 1-2 | Skin1 | TAGTGTTAGGAATTTAAGGGT | CCAAAATTAACAAAAACAA |
| (all T) | | TAGT (1) | AAAA (2) |
| NewSkin2 | Skin3 | TGTATTTTAGGTTTTTATATTT | AAACAAAACTACTATCAAA |
| (all C) | | TTATTA (3) | TCCTTC (4) |
| NewSkin10 | Skin2 | TTGTTAGAAGTAGTTAAATTA | CTAACCCCCAAATACAAAC |
| (all C) | | TTTTTT (5) | C (6) |
| Skin2 (all C) | Skin4 | GGTTTGTGATTTTGTAAGTATT | ACCCACTATCCCTCTAAAC |
| | | AA (7) | TC (8) |
| SLC22A5 | Skin5 | ATTATTAGGGAGAGGAAATAT | CTAACCCACCAAACTCCAA |
| (all T) | | GTT (9) | A (10) |
| IGF2R (all | Liver1 | TGGGTGTTGTTATTTTGTTGA | CTACAAAAATACACACCCC |
| T) | | (11) | AA (12) |
| ITIH4 (all T) | Liver2 | ATAGTGAAGATGTTAGTTTGT | AACACACTTACCTAATAAC |
| | | TTTT (13) | CAAAC (14) |
| VTN2 (all T) | Liver3 | GGTATTTTGAAGAGGTAGGTT | ACCTAAATACCCCAAACTC |
| | | T (15) | AT (16) |
| cg17952661 | Liver4 | AGTTTTTTTATAATAGTTTTTT | ACACTAAAATTTCAAACAA |
| (all C) | | GTTAT (17) | AACTC (18) |
| GPAM (all | Liver5 | TTTTTTATTGTTTTAATGTTTTT | TAAACTCAATCCCCTAAAT |
| C) | | TAG (19) | ATCTAC (20) |
| C19orf35 | Colon1 | GGTATTGGGGATATTTTAGAG | CCTCCTAAACAACTCACCC |
| | | AG (21) | TAC (22) |
| cg09094964 | Colon2 | AGTAAAATGTTGGGTAGGTTT | TAAAATCATCTTAAAAATC |
| | | T (23) | AAATAC (24) |
| cg10900049 | Colon3 | TTGTGTTAGTAGAGAGAGGAA | CTTCAAAATACAAAACACT |
| | | AGAG (25) | CATCTC (26) |
| cg12462916 | Colon4 | GGAGGTTAAGGAGAGGGAGT | AAAAAAAAACTACCAAAA |
| | | (27) | CACC (28) |
| cg23460250 | Colon5 | AGTTTGGTTTGGGGTTTAGAG | CACACCCTCTCACACAAAC |
| | | (29) | AC (30) |
| ECH1 | Colon6 | GTTAGAAGGTATAGAAATAAT | TCTCCAAACTCTAAAAACC |
| | | TGTTAT (31) | CT (32) |
| hcol1 | Colon7 | GTTTTTTGTTTTTGTAGGTTGA | CTTCAAAATACAAAACACT |
| | | (33) | CATCT (34) |
| CPNE2 (all | Lung1 | TTTTTTATTTTTTGGGTATTTGT | TAAAAACACTCACATTCCA |
| T) | | (35) | ATAAA (36) |
| lungR_meth4 | Lung2 | GGTATAGTGATTAGGGGGTAG | AAAATAACTAAAACAAACC |
| (all C) | | TTAT (37) | CTACC (38) |
| ATP11A.R | Lung3 | GAGAAGTTAGGAGGAGAGTA | TTTACATTTTAAATTTTATC |
| (all T) | | GATA (39) | CC (40) |
| DGKD (all | Lung4 | TTTGTGTGAATAGAAAGATTT | AATATAACTCCACCCCAAA |
| T) | | TAGTT (41) | ATC (42) |
| lungR- | Lung5 | TGATAGATGAGGAAATTGAGG | AAAACAAACTTTAAAATCT |
| unmeth 1 (all | | TT (43) | AATCAAC (44) |
| lungR- | Lung6 | GGAAGTTTTGGTATGATTTTTT | ACTCTAATATAAACACCTA |
| unmeth3 (all | | (45) | ACAACC (46) |
| lungR- | Lung7 | TTTTTTTTGAGATGGGATTT | TAAAAAATTAAAATTACAA |
| unmeth4 (all | | (47) | TAAACC (48) |
| lungR_meth2 | Lung8 | GTAGTTGGGATTTAGAGAAGG | AACCCACAACCTAAAATCC |
| (all C) | | TT (49) | TAC (50) |
| lungR_meth3 | Lung9 | GGAATTTTGGAGGTTGTAGG | TTATCTTACTAATCATACTA |
| (all C) | | (51) | CTTCCC (52) |
| SOX2-OT | Lung10 | GGGGTTTTAATTTAGGGTTTA | AATTCACAAATTATTAACA |
| (all C) | | G (53) | AACACC (54) |
| S5-unMe- | Lung11 | TAGAGTATTGGTTTGAAGATT | TATCACAACCACACATAAA |
| L1/R1 (all T) | | TGT (55) | CAAC (56) |

Clinical assessment of HISCT patients in the chronic setting: To assess the utility of cfDNA for detecting organ damage in chronic GVHD, 101 plasma samples from 101 individuals were collected. These individuals were more than 100 days post allogeneic stem cell transplantation and arrived for clinical follow up at the BMT day care unit at Hadassah medical center. Patients agreeing to participate, regardless of their cGVHD status, signed an informed consent. Upon each visit blood was drawn for regular blood tests (extra 10 ml of blood was drawn for cfDNA analysis) and the patient underwent a full assessment by the treating physician which included cGVHD grading according to the 2014 National Institute of Health (NIH) criteria. During the course of this 38-month study, 65 patients were diagnosed at any point with clinically evident cGVHD, while 36 were not found to have clinical signs of cGVHD.

Patient Characteristics: The median age of patients was 47 years. Sixty five percent of the patients were males. The majority of patients were transplanted due to acute myeloid leukemia (57%), had a matched sibling (63%), were treated with a myeloablative conditioning regimen (64%) and received stem cells withdrawn from peripheral blood (PBSC, 92%). Most of the patients (55%) underwent a transplant from a matched gender while 25% were transplanted from a mismatched donor gender, in a female to male direction.

Fifty-seven percent of the 101 samples were collected from patients with a history of acute GVHD. None had signs of overlap (both acute and chronic) GVHD at the time of sampling. One patient developed liver GVHD one month post Donor Lymphocyte Infusion (DLI). The median time from transplantation was 783 days (range 101-7878 days). Half of the samples (49) were taken from patients receiving one or more immunosuppressive agents at the time of collection. Only seven patients had evidence of CMV viremia at the time of collection. One patient had biopsy proven colitis, which did not show CMV inclusion bodies while none of the remaining six had any evidence for CMV disease. Four patients were treated for CMV infection. Eleven patients had a positive EBV-PCR in peripheral blood (with a median of 300 copies/ml), none of which was clinically significant. One patient was positive in the upper respiratory tract for RSV and one for influenza. One patient had staphylococcus epidermis bacteremia. Chimerism levels were routinely monitored. Ninety eight percent of the samples were obtained from patients with a blood driven STR assay indicating 100% donor-derived hematopoietic cells. Two samples exhibited a donor chimerism ranging from 88% to 92%, precluding analysis of the relationship between degree of chimerism, cfDNA methylation profiles and a potential relapse. None of the samples were taken at the time of relapse.

Statistical analysis: Assessment of cfDNA plasma levels in healthy controls versus allogeneic transplanted patients with and without clinical signs of cGVHD was performed using nonparametric, unpaired, Mann Whitney test. Analyses were performed using GraphPad Prism (version 10.0.1) and results were considered statistically significant for p-values of ≤0.05.

Machine learning was used to evaluate the predictive power of both cfDNA and biochemical measurements in relation to clinical evident cGVHD. Multivariate logistic regression (MLR), XG boost and random forest (RF) classifiers were compared on the data set. MLR, XGboost, and RF had an average accuracy of 0.74, 0.67, and 0.65, respectively, by Repeated-K-fold cross-validation (K=5) with a standard deviation of 0.23, 0.22, and 0.3, respectively. As the MLR model had both higher accuracy with similarly robust results by cross-validation, MLR was applied for further analyses. Furthermore, MLR emerges as the most fitting estimator based on the following considerations:
It is anticipated that GVHD will consistently increase the levels of measured markers, signifying increased cell death. Hence, a monotonous model which consistently increases in response to changes in its features, should be appropriate;
The size of the data does not support models with a large number of parameters—MLR bears a single parameter per measurement, reducing the risk of overfitting; and MLR inference naturally provides a probability score.

It was hypothesized that measurements of cfDNA and blood biochemical values possess significant predictive potential for the presence of cGVHD. Shapley values were leveraged to gauge the magnitude of the predictive capability of each feature. This latter technique offers a principled approach to feature selection, promoting enhanced performance with reduced overfitting. Since higher cell-free DNA levels are expected to indicate cGVHD, the parameter space of the model was constrained to be non-negative for all coefficients. The performance was compared to an unconstrained optimization in order to explore the overfitting potential of the model. A total of 93 samples (for which data was available for all parameters) were used for the analysis. Shapley analysis (Lundberg and Lee, “A unified approach to interpretting model predictions”, Advances in Neural Information Processing Systems. 2017:4765-74, hereby incorporated by reference in its entirety) was performed on a collection of 17 features (consisting of GGT, ALP, ALT, AST, TBil, Total cfDNA level [presented in ng/ml], and organ specific cfDNA: cfSkin, cfLung, cfGI, cfLiver, cfNeutrophils, cfMonocytes, cfEosinophils, cfB cells, cfT cells, cfCD8 cells, cfTregs cell). Next, to robustly validate the predictive potential of the features, Repeated-K-Fold cross-validation was utilized. Repeated 5-fold cross-validations was conducted across the feature sets given a positive coefficient (constrained optimization). Each set, labeled n=1, . . . , 17, consists of the highest-ranking n features, meaning, set n=1 is the single top-ranking feature, set n=2 consists of the 2 top ranking features, set n=3 of the 3 top ranking features and so on. The metrics (Specificity, NPV, PPV, AUC and Precision) for each n were calculated. The selection of the best feature set was determined based on those achieving the highest AUC (Area Under the Curve) and demonstrating favorable performance across other metrics. Recall, specificity, AUC, NPV and PPV of logistic regression models trained using only the best feature set was calculated. A comparison between cfDNA features compared to blood biochemical features and to the combination of both (meaning the entire set) was performed. All analyses were performed using Python 3.10.

Using the model, accuracy ([(TP+ TN)/Total testing samples]×100%), specificity ([TP/(TP+FN)]×100%), sensitivity([TN/(TN+FP)]×100%, positive predictive value (PPV) ([TN/(TN+FN)]×100%) and negative predictive value (NPV)/precision ([TP/(TP+FP)]×100%) were measured.

Graphical representation of the tradeoff between specificity and sensitivity was done using the receiver operating characteristics curve (ROC). Area under the curve (AUC) was calculated in order to determine the ability of the classifier to distinguish positive and negative results. Spearman rank correlation was used to determine the significance of correlation between each pair of variables and other parameters.

### Example 1: DNA Methylation Markers for Targeted Assessment of cGVHD-Relevant Tissue Damage

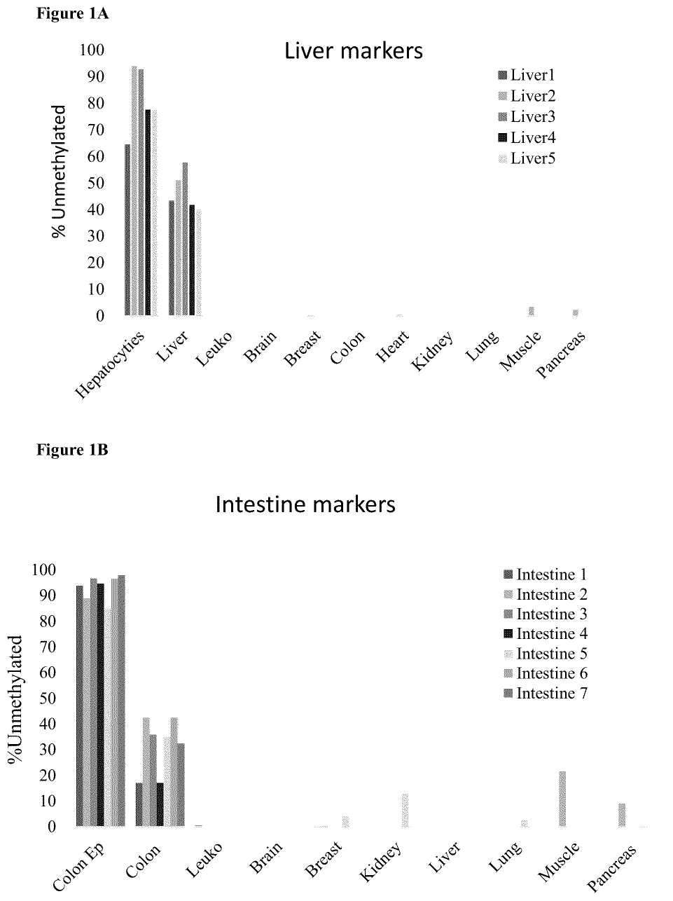

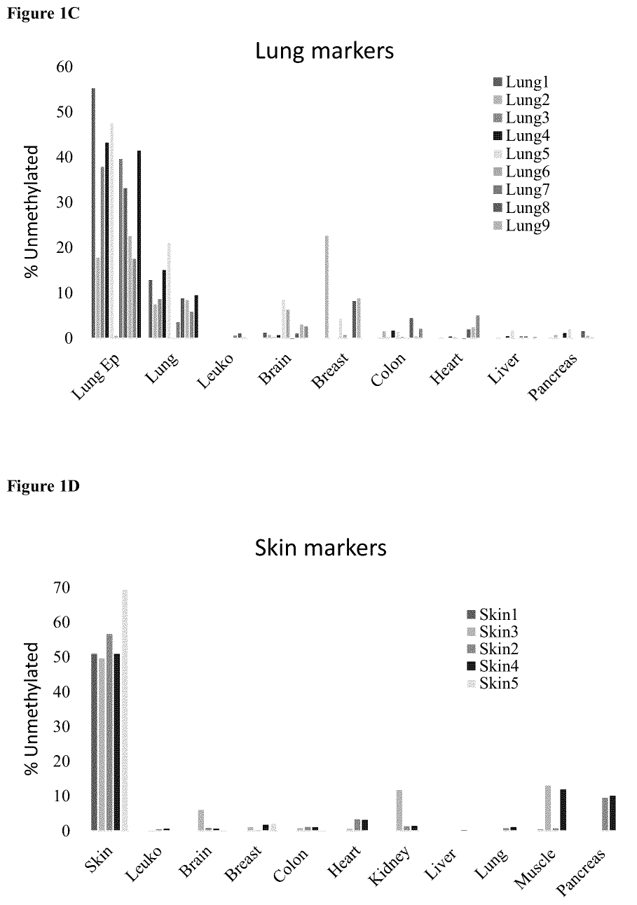

By comparing publicly available methylomes of specific human tissues (see Moss et al., “Comprehensive human cell-type methylation atlas reveals origins of circulating cell-free DNA in health and disease”, Nat. Comm., 2018; 9(1):5068) genomic loci containing CpG sites have been identified that are uniquely unmethylated in specific tissues or cell types, relevant to cGVHD: These included hepatocyte (5 markers), skin (5 markers), lung epithelial cells (10 markers), and intestinal epithelial cells (7 markers). Informative loci were selected from Moss et al., 2018, “Comprehensive human cell-type methylation atlas reveals origins of circulating cell-free DNA in health and disease”, Nat. Commun., 9:5068, herein incorporated by reference in its entirety. Table 1 provides the primers for amplification of the methylation marker sequences used herein. Multiplex PCR cocktails have been designed to amplify all these loci from genomic DNA after bisulfite conversion and the products have been sequenced to determine the fraction of unmethylated DNA molecules present in the starting material. FIG. 1 shows the fraction of methylation blocks from each marker locus that were unmethylated in the indicated samples. Molecules containing multiple unmethylated CpG sites could be assigned with extreme specificity to a given tissue of origin. Also, genomic DNA from specific tissues was spiked into genomic DNA of leukocytes to determine assay sensitivity and linearity and it was found that as little as 0.5% from the target tissue could be robustly identified when present in a mixture (not shown). These findings establish a cocktail of DNA methylation markers that can be used to identify DNA derived from the liver, skin, lungs and intestine with extreme specificity and sensitivity. Methylation markers where also designed that are specific to selected immune and inflammatory cell types: neutrophils, eosinophils, monocytes, B lymphocytes and T lymphocytes (including CD8+ and regulatory T cells); all of which showed extreme specificity and sensitivity.

### Example 2: Elevated cfDNA Levels in HCT Patients with and without Clinical GVHD

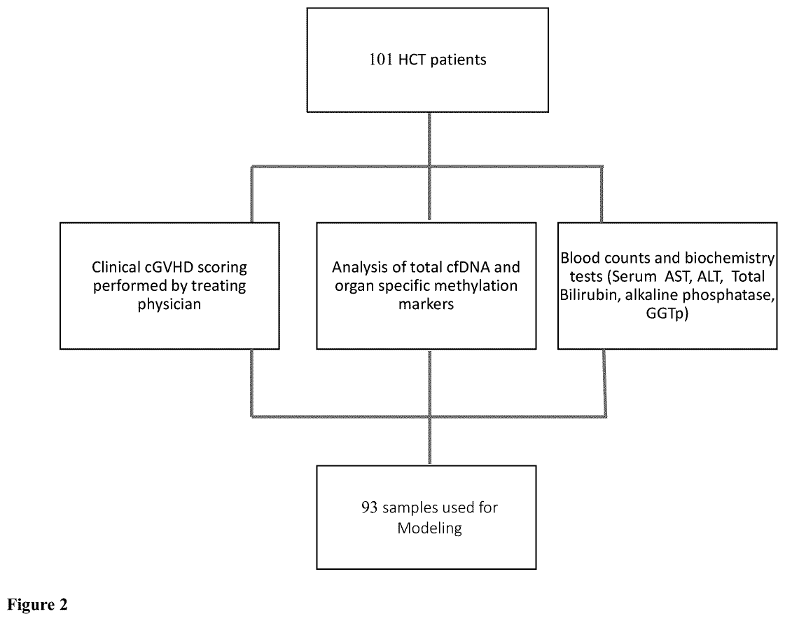

The overall scheme of the experiment is shown in FIG. 2. A total of 101 HCT patients were recruited. Blood samples were obtained and clinical cGVHD score as well as blood counts and standard blood biochemistry were recorded. Plasma cfDNA concentration and methylation patterns were determined and compared to the clinical and biochemistry data, and then a model for inference of cGVHD based on cfDNA parameters combined with blood biochemistry markers was developed and validated. The characteristics of the 101 recruited patients and samples are detailed in Tables 2 and 3 and in the Methods section.

TABLE 2
Sample characteristics

| | Number of Patients | |
|---|---|---|
| Variables | (Total = 101) (%) | |
| Age (Years) | | |
| Median (Range) | 47 | (18-74) |
| Gender | | |
| Male | 65 | (64.4%) |
| Female | 36 | (35.6%) |
| Hematological Disease | | |
| AML | 57 | (56.4%) |
| ALL | 18 | (17.9%) |
| LYMPHOMA | 11 | (10.9%) |
| MDS | 7 | (6.9%) |
| OTHER | 8 | (7.9%) |
| Donor Type | | |
| MSD | 64 | (63.4%) |
| MRD | 4 | (3.9%) |
| MUD | 28 | (27.8%) |
| HAPLO | 4 | (3.9%) |
| CORD BLOOD | 1 | (1%) |
| Matching | | |
| Match | 77 | (76.3%) |
| Mismatch | 19 | (18.8%) |
| UK | 5 | (4.9%) |
| Stem Cells Source | | |
| PBSC | 93 | (92.1%) |
| BM | 7 | (6.9%) |
| UK | 1 | (1%) |
| Conditioning Intensity | | |
| MA | 65 | (64.4%) |
| RIC | 16 | (15.8%) |
| NMA | 19 | (18.8%) |
| UK | 1 | (1%) |
| GVHD Prophylaxis | | |
| CSA | 16 | (15.8%) |
| CSA + MTX | 18 | (17.9%) |
| CSA + MMF | 61 | (60.4%) |
| PTCY + TAC + MMF | 5 | (4.9%) |
| UK | 1 | (1%) |
| Gender Match | | |
| M TO M | 45 | (44.6%) |
| F TO F | 11 | (10.9%) |
| M TO F | 19 | (18.7%) |
| F TO M | 25 | (24.8%) |
| UK | 1 | (1%) |
| ATG | | |
| Yes | 51 | (50.5%) |
| No | 49 | (48.5%) |
| UK | 1 | (1%) |

TABLE 3
Post transplantation patients characteristics

| | Number Of Samples | |
|---|---|---|
| Variables | (Total = 101) (%) | |
| Acute GVHD | | |
| No | 42 | (41.6%) |
| Yes | 58 | (57.4%) |
| UK | 1 | (1%) |
| Chronic GVHD | | |
| No | 36 | (35.6%) |
| Yes | 65 | (64.3%) |
| Chronic GVHD Grade | (TOTAL = 65) (%) | |
| Mild | 27 | (41.5%) |
| Moderate | 22 | (33.9%) |
| Severe | (24.6%)16 | |
| Prednisone >10 mg | (Total = 101) (%) | |
| No | 86 | (85.1%) |
| Yes | 15 | (14.9%) |
| Immunosuppressive Agent at | (Total = 47) (%) | |
| time of sampling | | |
| CSA | 24 | (51.1%) |
| Tacrolimus | 18 | (38.3%) |
| Sirolimus | 3 | (6.4%) |
| Ruxolitinib | 6 | (12.8%) |
| MTX | 1 | (2.1%) |
| ECP (2 weeks prior) | 2 | (4.3%) |
| Chronic Organ Involvement | (TOTAL = 65) (%) | |
| Skin | 29 | (44.6%) |
| Sclerodermatous | 14 | (21.5%) |
| Lung | 22 | (33.8%) |
| Liver | 12 | (18.5%) |
| GI | 2 | (3.1%) |
| Eyes | 38 | (58.5%) |
| Mouth | 23 | (35.4%) |
| Genital | 7 | (10.8%) |
| Joints And Fascia | 12 | (18.5%) |

Total and tissue-specific cfDNA concentration in samples from healthy individuals (median age 37 years old (range 24-68), 58% females), samples from HCT patients that had no evidence of cGVHD at the time of sampling, and samples from HCT patients defined by their treating physician as having clinically evident cGVHD were compared. The NIH 2014 criteria was used by the treating physician for defining disease severity (mild, moderate and severe) and organ scoring (0-3).

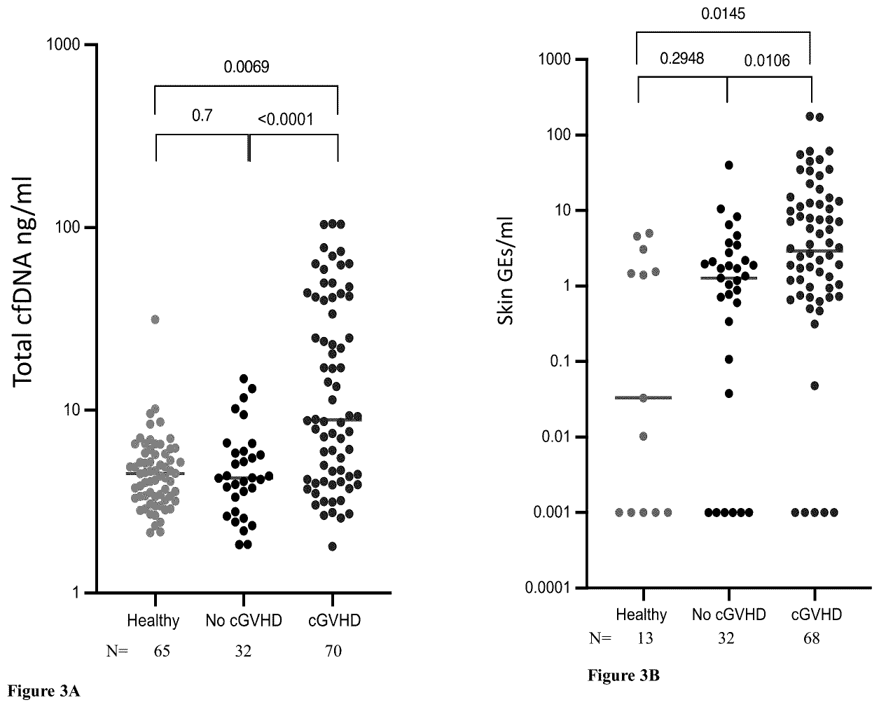

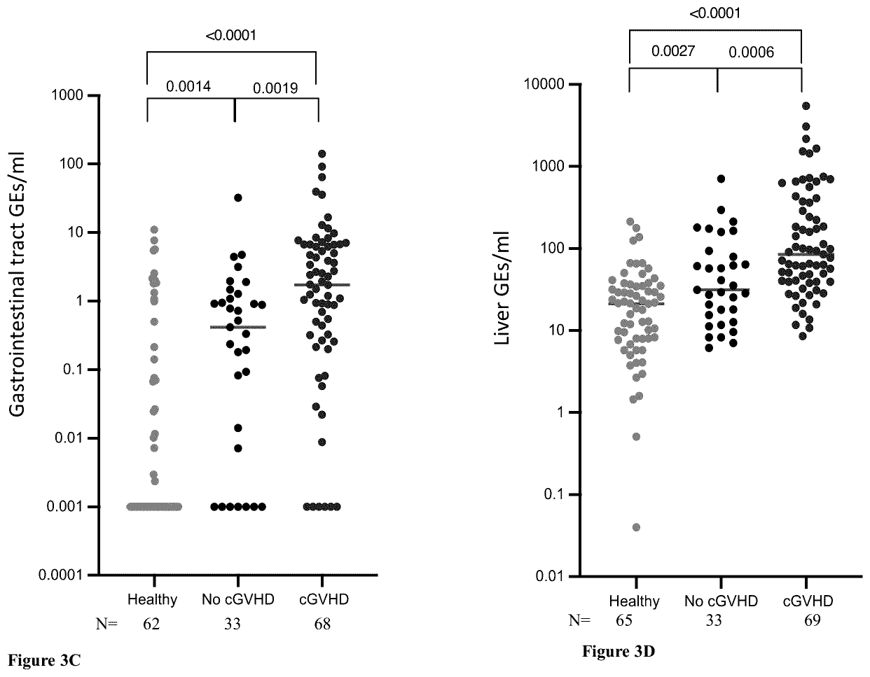

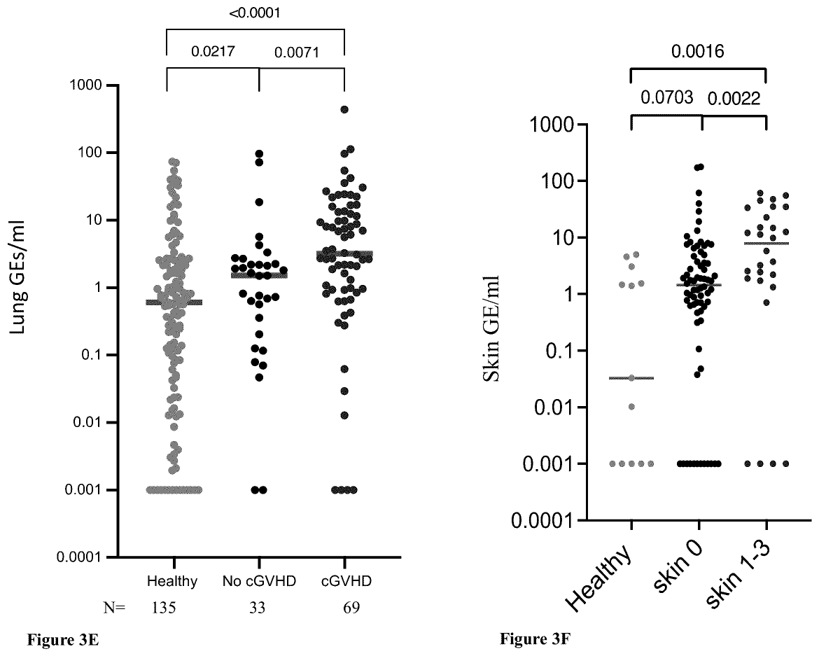

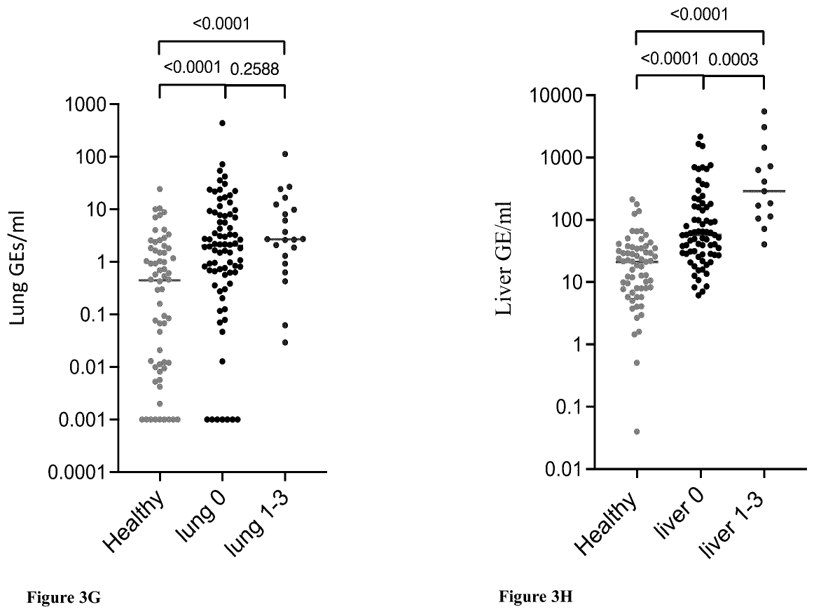

Analyzing 101 samples from 101 patients, HCT patients suffering from cGVHD (in any organ) had statistically significant higher concentrations of total cfDNA compared to HCT patients with no clinical evidence of cGVHD (p<0.0001) (FIG. 3A). This is directly contradictory to previous reports (see, Duque-Alfonso et al., 2017, “Cell-free DNA characteristics and chimerism analysis in patients after allogeneic cell transplantation”, Clin. Biochem., February; 54:137-141). Total cfDNA levels in HCT patients were similar to those in healthy controls (p=0.7).

cfDNA signals from skin (p=0.0106, FIG. 3B), intestine (p=0.0019, FIG. 3C), liver (p=0.0006, FIG. 3D) and lungs (p=0.0071, FIG. 3E) were also significantly higher in the clinically evident cGVHD group compared to the group of patients who did not meet the NIH 2014 criteria for cGVHD. In addition, the concentration of cfDNA originating from gastrointestinal tract (GI), liver and lung was significantly higher in patients who underwent HCT with no evidence of clinical GVHD compared to healthy controls (p=0.0014, p=0.0027, and p=0.0217, respectively) (FIG. 3C-3E). Moreover, cfDNA originating from skin and liver significantly correlated with organ specific clinical GVHD presence (score 0 versus score 1-3) (p=0.0022, p=0.0003, FIG. 3F and FIG. 3H, respectively). Interestingly, HCT patients with and without lung cGVHD (score 0 vs 1-3) had significantly higher levels of lung cfDNA compared with healthy controls (p<0.0001), but lung cfDNA did not correlate with the presence of clinical lung score (FIG. 3G).

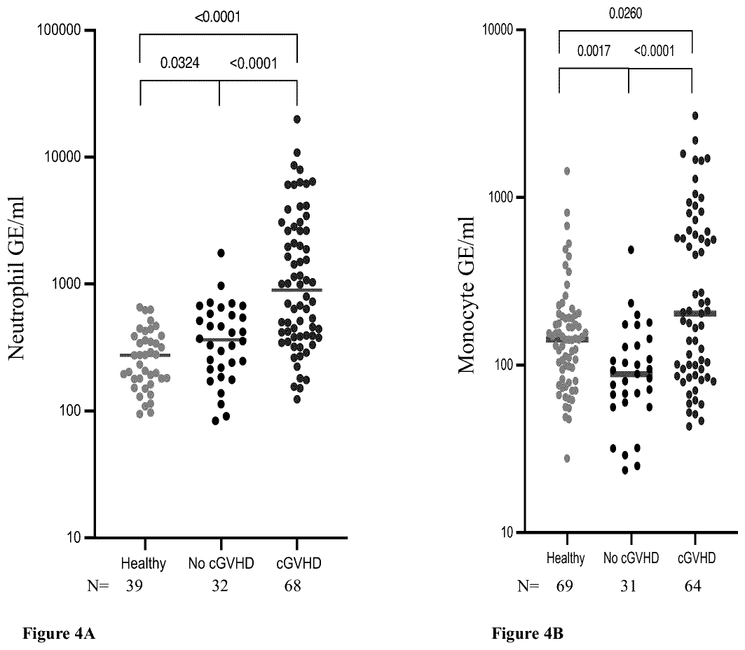

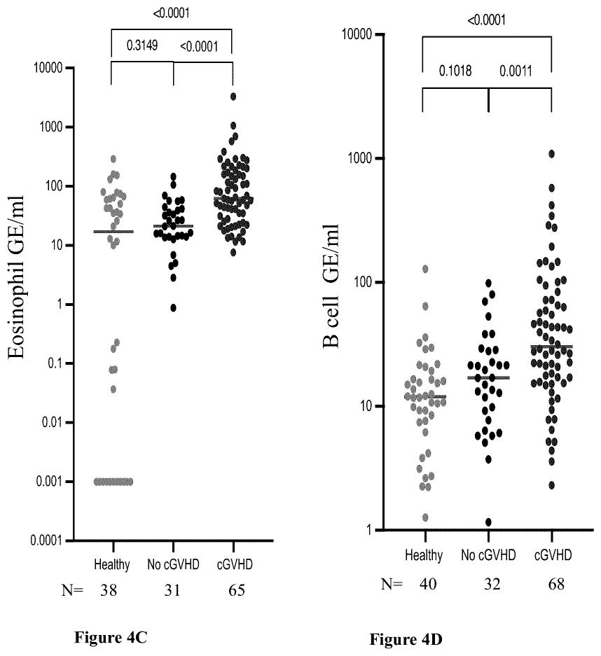

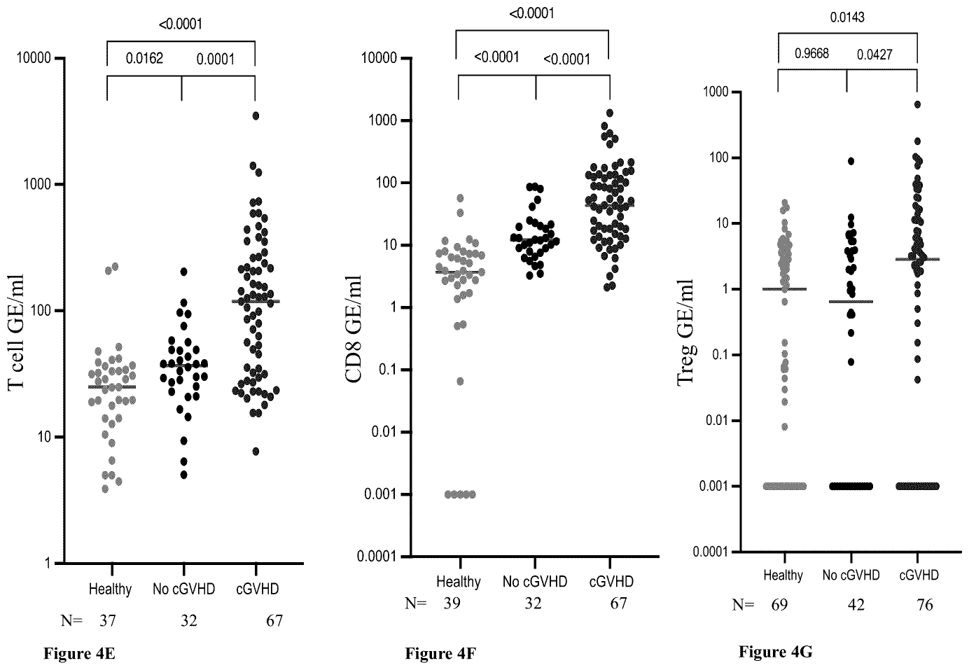

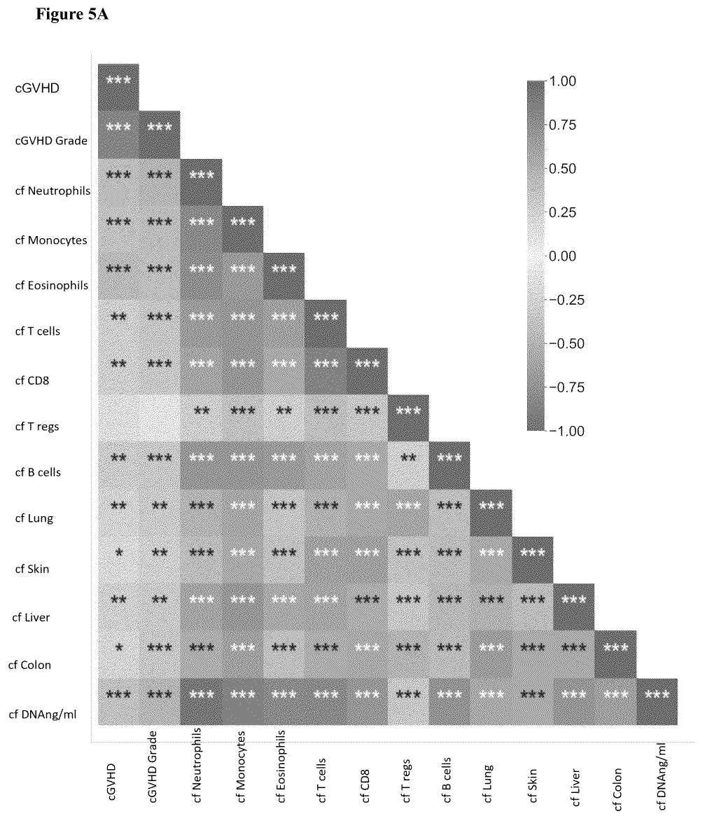

Analysis of immune-derived cfDNA showed a significantly higher concentration of cfDNA originating from neutrophils, monocytes, eosinophils, and B and T lymphocytes in HCT patients diagnosed with clinical cGVHD as compared to those who were not (FIG. 4A-4G). cfDNA from neutrophils, T cells and CD8 positive cells was elevated in HCT patients that have no clinical cGVHD as compared to healthy volunteers (FIG. 4A, 4E-4F).

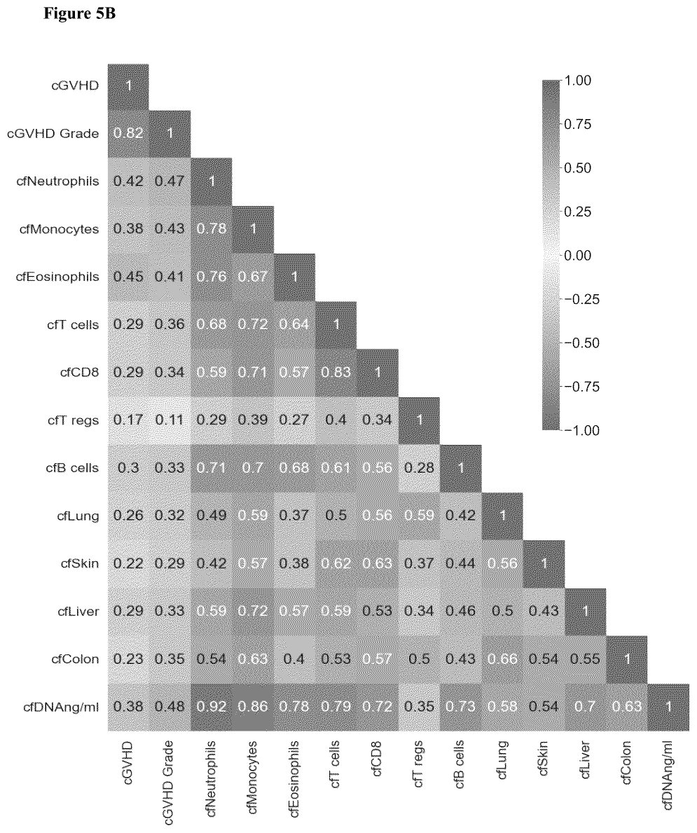

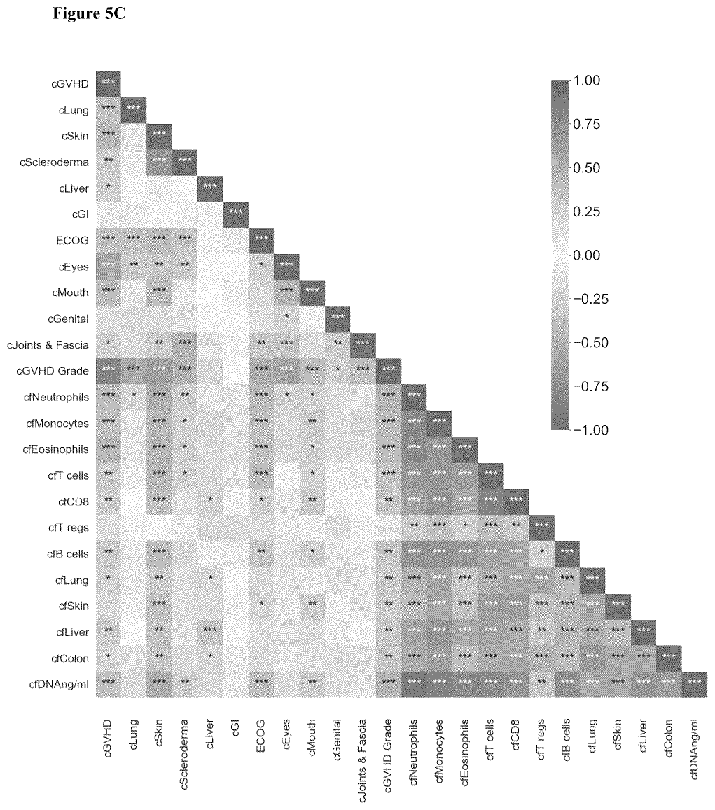

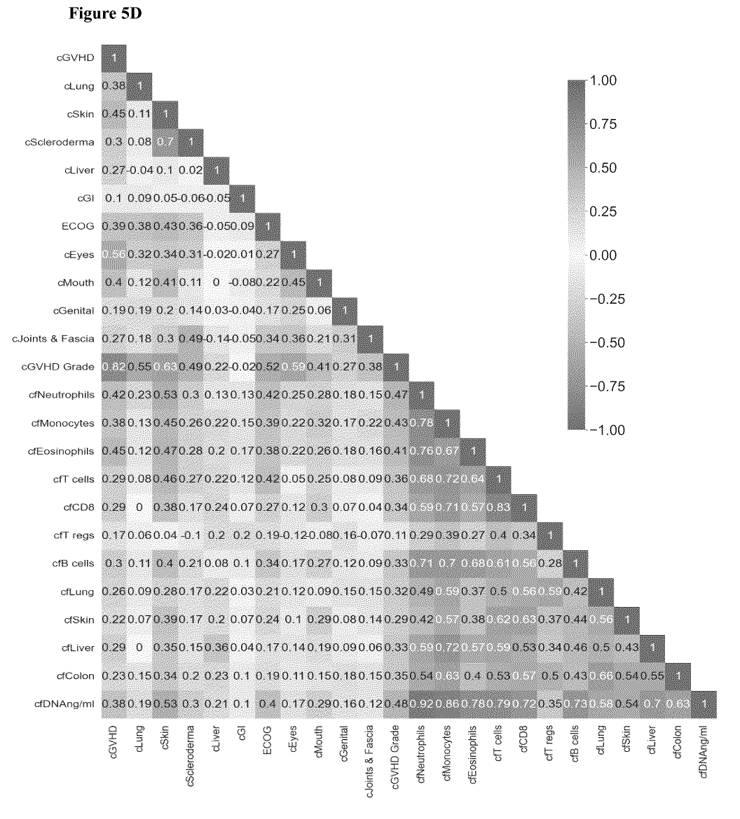

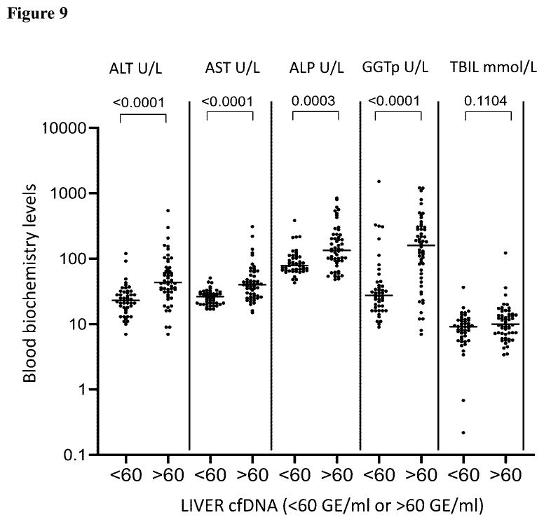

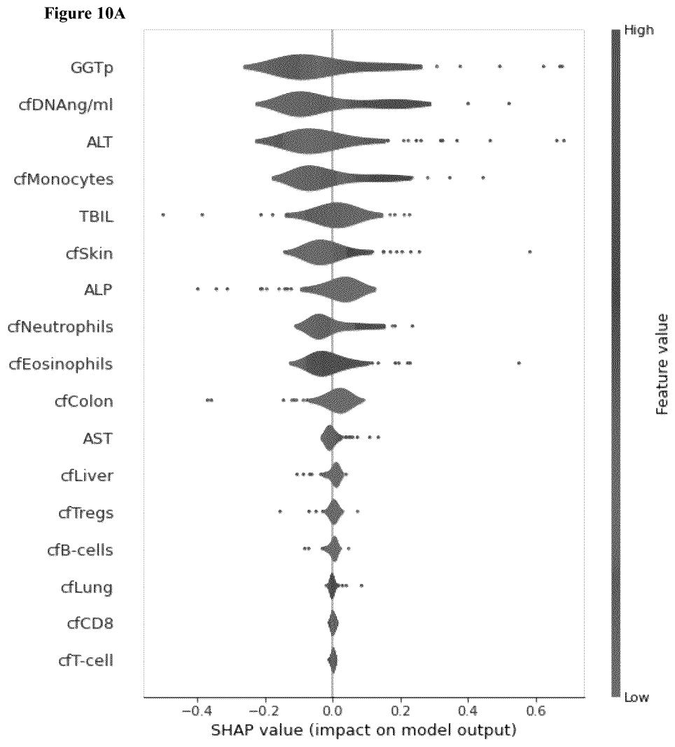

Next, correlations between cfDNA parameters and cGVHD clinical scores among HCT patients were sought. A correlation matrix was produced for all 101 plasma samples for which all tested parameters were available. cfDNA parameters were highly correlated internally (for example, samples with high concentration of total cfDNA tended to also have high levels of organ specific cfDNA) (FIG. 5A-5B), and there was a significant internal correlation among cGVHD clinical scores (between cGVHD severity assessment and specific organ grading) (FIG. 5C-5D). Moreover, a significant correlation between clinical cGVHD severity assessment and total cfDNA as well as organ specific cfDNA levels was found (FIGS. 5A, 5C-5D and 9).

### Example 3: A Combined Score for Blood-Based Detection of cGVHD

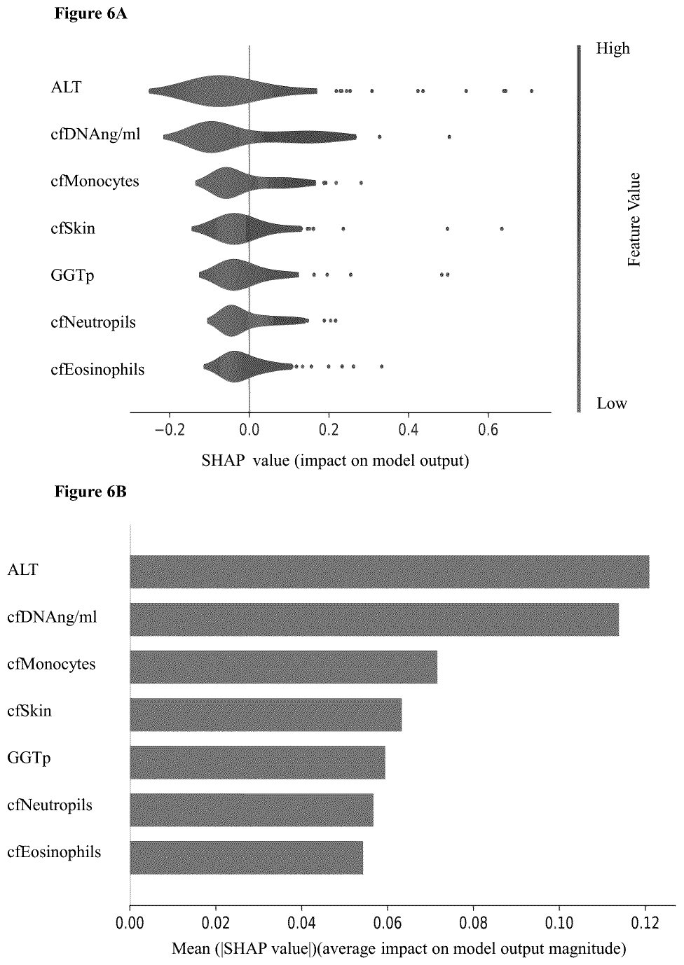

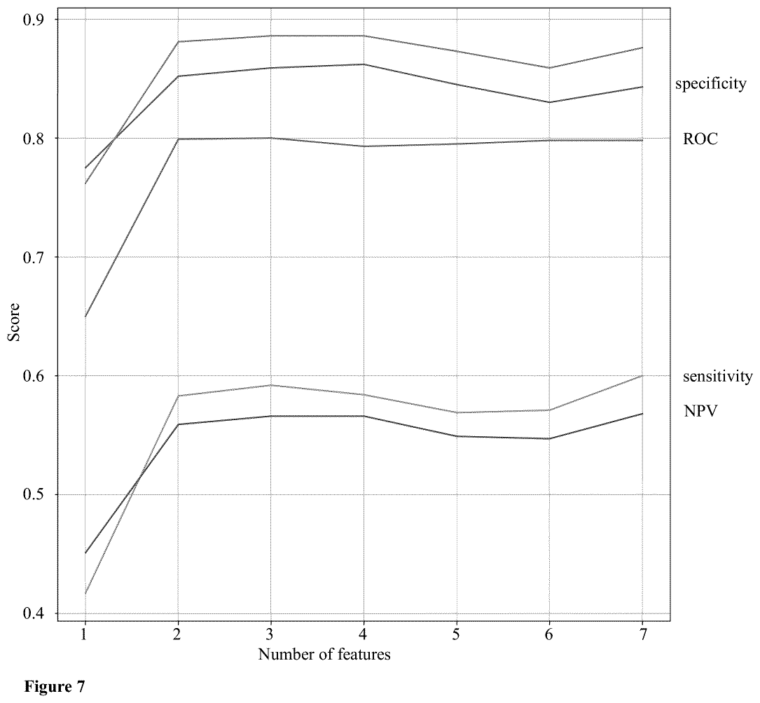

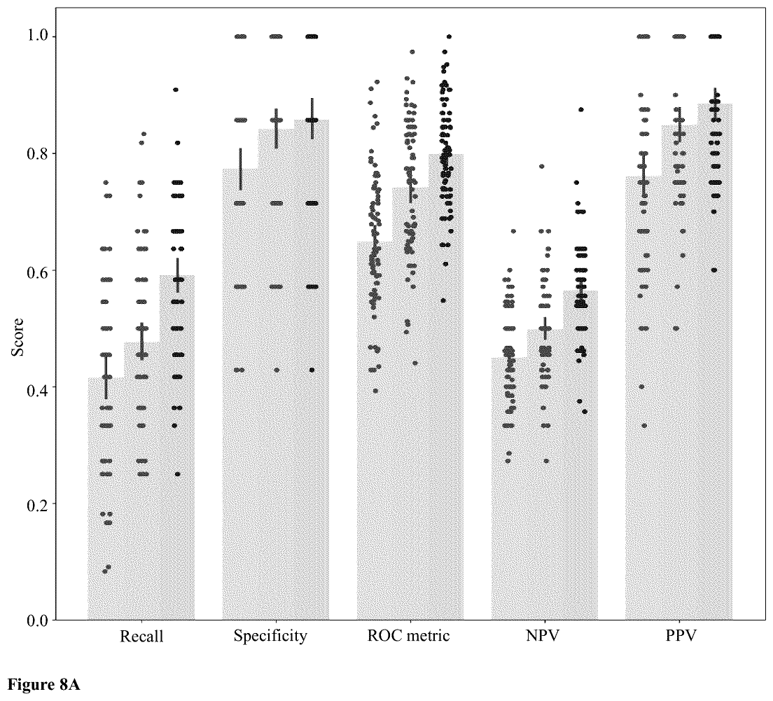

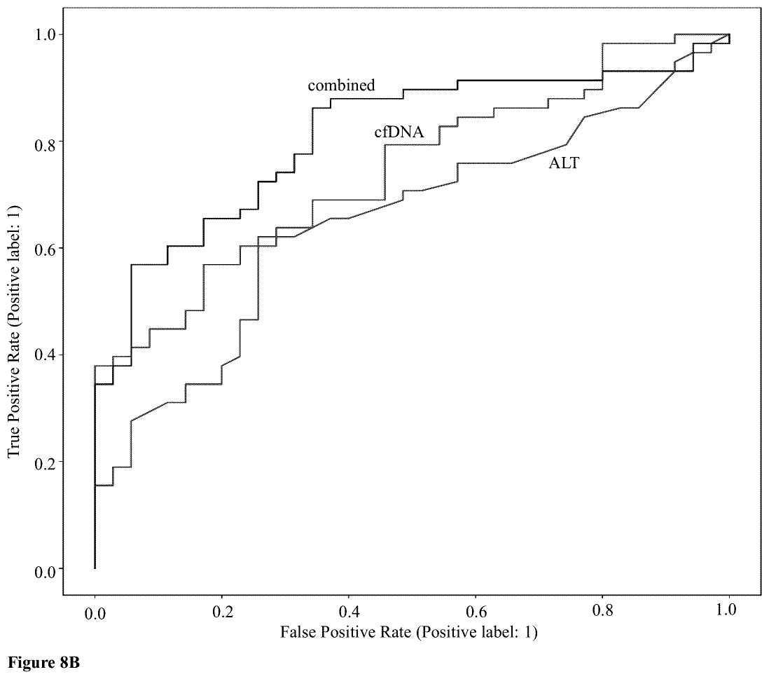

An attempt was made to use machine learning to create a model which can aid the treating physician in predicting the likelihood that a patient has active cGVHD. Employing Shapley analysis on all 17 features (detailed in the Materials and Methods section) yielded positive Shapley values for 7 features (alanine transaminase (ALT), total cfDNA, cfDNA of monocytes, cfDNA of skin, gamma glutamyl transpeptidase (GGTp), cfDNA of neutrophils and cfDNA of eosinophils), which are presented as a distribution graph in FIG. 6A. The average absolute SHAP value for each individual feature is provided in FIG. 6B. 5-fold cross-validations were repeated across these 7 feature sets, starting from the feature having the highest value (ALT) and sequentially adding the next feature in line (ALT and total cfDNA; ALT, total cfDNA, cfDNA of monocytes and so on). The metrics (specificity, Negative predictive value (NPV), positive predictive value (PPV), AUC and precision) for each number of features selected are illustrated in FIG. 7. Notably, the three first features maximize the AUC, and display favorable behavior across the other metrics. Therefore, these three features (consisting of ALT, total cfDNA and cfDNA of monocytes) were selected as the optimal feature set. Recall, specificity, AUC, NPV and PPV of logistic regression models trained using only ALT, only cfDNA features (total cfDNA and cfDNA of monocytes) and all three features are shown in FIG. 8A. The ROC curves of these models are shown in FIG. 8B.

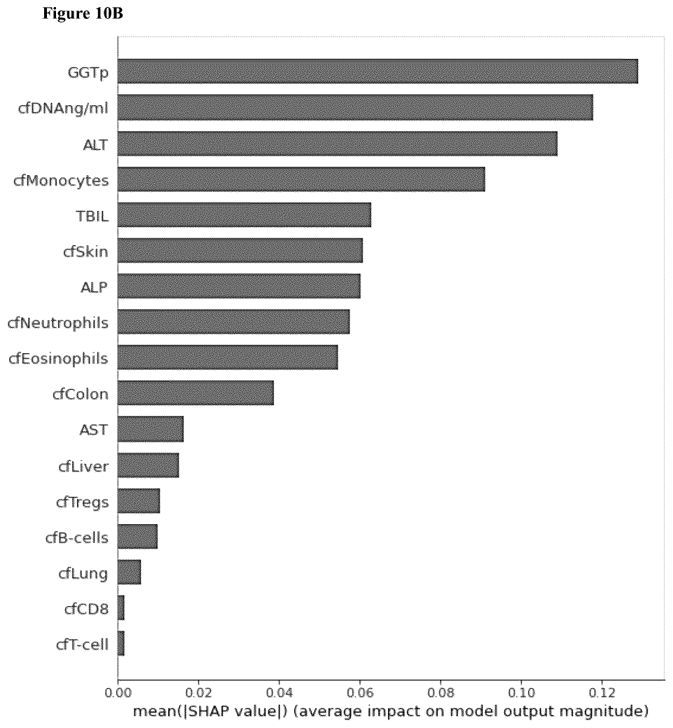

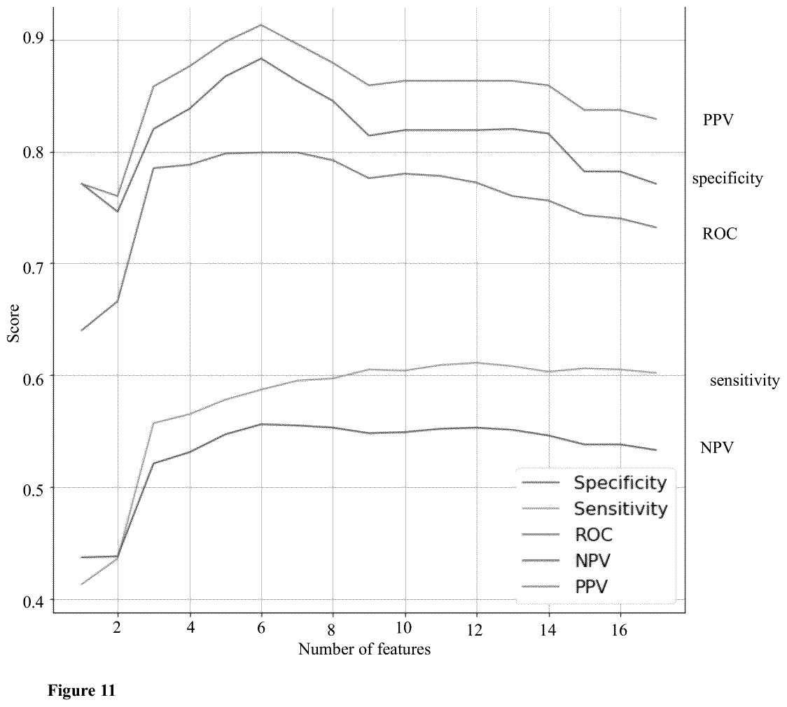

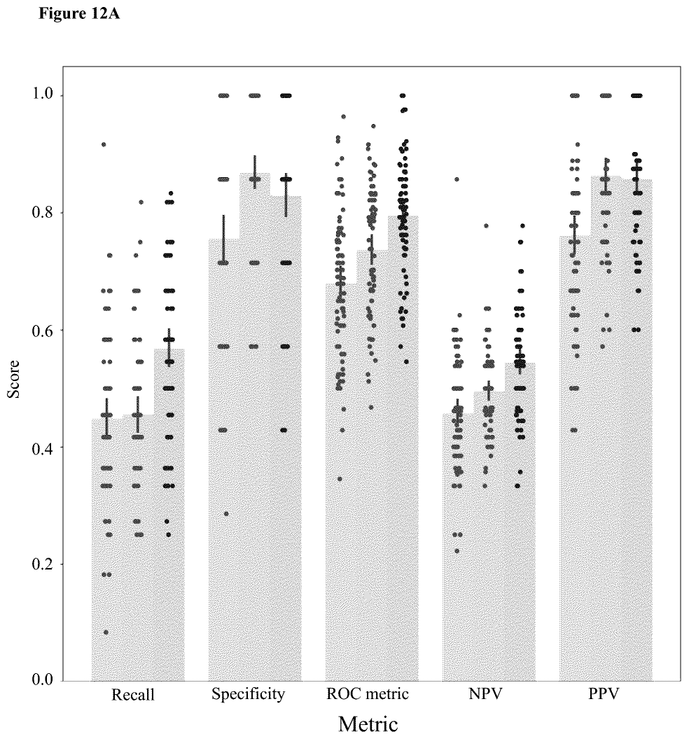

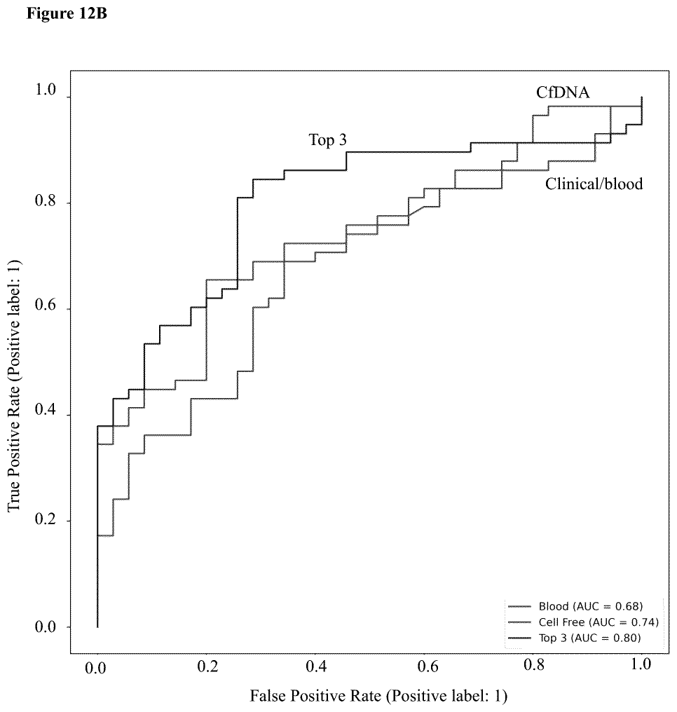

Finally, the performances of the models were compared to the exact equivalent set of models, where, instead of using a constrained optimization, an unconstrained optimization (allowing negative coefficients) was used. Shapley values were calculated for all 17 features (FIG. 10A-10B). The metrics (specificity, NPV, PPV, AUC and precision) for each n are illustrated in FIG. 11. Favorable behavior across all metrics was reached at n=6 (ALT, GGTp, total cfDNA, cfMonocytes, cfEosinophils, and ALP) and repeated 5-fold cross-validation was performed to compare the recall, specificity, AUC, NPV and PPV of logistic regression models trained using these features (FIG. 12A), as well as the ROC curves of these models (FIG. 12B).

Evidently, both constrained and unconstrained optimization techniques demonstrate comparable performance, suggesting minimal overfitting with either optimization technique. Moreover, emphasizing the high predictive capability of a small set of features, consisting of biochemical as well as cfDNA measurements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of diagnosing graft versus host disease (GVHD) in a subject that has undergone a hematopoietic stem cell transplant (HCT) from a donor, the method comprising receiving a measurement of cell-free DNA (cfDNA) quantity in a fluid sample from said subject, wherein a cfDNA quantity above a predetermined threshold indicates said subject has GVHD, thereby diagnosing GVHD.

2. The method of claim 1, wherein said HCT occurred at least 100 days before said sample was taken.

3. The method of claim 1, wherein said GVHD is chronic GVHD.

4. The method of claim 1, further comprising extracting said fluid sample from said subject, isolating cfDNA from said extracted sample and quantifying said isolated cfDNA.

5. The method of claim 1, wherein said fluid is selected from peripheral blood, plasma and serum.

6. The method of claim 1, wherein said cfDNA quantity is the total quantity of cfDNA in said fluid sample.

7. The method of claim 1, wherein said cfDNA comprises cfDNA from said subject and cfDNA from said donor.

8. The method of claim 1, wherein said method does not comprise isolating cfDNA only from said subject.

9. The method of claim 1, wherein said predetermined threshold is selected from: the cfDNA quantity in a fluid sample from a healthy control subject or from a population of healthy control subjects and the cfDNA quantity in a fluid sample from a control subject that underwent an HCT and does not have GVHD.

10. The method of claim 1, wherein said cfDNA is cfDNA originating from a specific tissue or cell type and said specific tissue or cell type is selected from skin, lung, liver, intestine, B cells, T cells, CD8 positive T cells, eosinophils, monocytes, neutrophils and T regulatory cells (Tregs).

11. The method of claim 10, wherein said specific tissue or cell type is selected from liver, B cells, T cells, CD8 positive T cells, eosinophils, monocytes, neutrophils and Tregs.

12. The method of claim 10, wherein the origin of said cfDNA was determined by
a. analyzing methylation of CpGs at an informative locus within said cfDNA, wherein said informative locus is uniquely methylated or unmethylated within said specific tissue or cell type so as to allow unique identification of the origin of said cfDNA based on the methylation status of said informative locus; or
b. chromatin immunoprecipitation sequencing (ChIP-Seq) and correlating binding of a cfDNA-associated protein with an informative locus within said cfDNA, wherein if said cfDNA-associated protein is indicative of active transcription said informative locus is uniquely actively transcribed within said specific tissue or cell type, and if said cfDNA-associated protein is indicative of silenced transcription said informative locus is uniquely silenced within said specific tissue or cell type, so as to allow unique identification of the origin of said cfDNA based on the protein associated with said informative locus.

13. The method of claim 12, wherein said measurement of cfDNA was generated by performing methylation sensitive sequencing on said cfDNA to produce the nucleotide sequence of said cfDNA including the methylation status of each cytosine in said nucleotide sequence and assigning sequencing reads as originating from said specific tissue or cell type based on said reads' nucleotide sequence and methylation status.

14. A method of classifying a sample as originating from a subject suffering from GVHD or a subject not suffering from GVHD, the method comprising:
a. receiving measurements of at least two parameters in a fluid sample, wherein said parameters comprise at least one clinical parameter and at least one cfDNA parameter, and wherein said clinical parameter is selected from the group consisting of: liver enzyme level, white blood cell (WBC) count, and hemoglobin level and said cfDNA parameter is selected from total cfDNA quantity and quantity of cfDNA from a specific tissue or cell type;
b. applying a trained machine learning (ML) model to said at least two received parameters; and
c. treating a subject diagnosed with GVHD with an anti-GVHD therapeutic agent or tapering immunosuppression treatment to a subject that is not diagnosed with GVHD;
thereby classifying a sample.

15. The method of claim 14, wherein said method is a method of diagnosing GVHD in a subject in need thereof, said sample is from said subject, said ML model was trained on a training set comprising said at least two parameters in subjects suffering and not suffering from GVHD and said ML model outputs a diagnosis of having GVHD or not having GVHD.

16. The method of claim 14, wherein said liver enzymes are selected from aspartate transaminase (AST), alanine transaminase (ALT), alkaline phosphatase (ALP) and gamma-glutamyl transpeptidase (GGTp), said cfDNA parameter is selected from: total cfDNA quantity, cfDNA quantity from neutrophils, cfDNA quantity from monocytes, cfDNA quantity from eosinophils, cfDNA from Treg cells, cfDNA quantity from T cells, cfDNA quantity from CD8 cells, cfDNA quantity from B cells, cfDNA quantity from skin, cfDNA quantity from intestine, cfDNA quantity from lung and cfDNA quantity from liver or both.

17. The method of claim 14, wherein said at least two parameters comprise ALT level and total cfDNA quantity.

18. The method of claim 17, wherein said at least two parameters comprise ALT level, total cfDNA quantity and cfDNA quantity from monocytes.

19. A method comprising:
training a machine learning model to predict the presence of graft versus host disease (GVHD) in a subject, on a training set, the method comprising:
i. extracting cfDNA from a fluid sample;
ii. quantifying the amount of cfDNA in said fluid sample; and
iii. determining at least one clinical parameter in said fluid sample;
wherein said training set is generated by labeling cfDNA quantities in samples and at least one clinical parameter as coming from a subject that suffers from GVHD or does not suffer from GVHD and compiling a plurality of cfDNA quantities, clinical parameters and their labels together to form said training set, wherein said plurality comprises labels of both subjects that suffer from GVHD and subjects that do not suffer from GVHD and wherein said clinical parameter is selected from the group consisting of: liver enzyme level, white blood cell (WBC) count and hemoglobin level.

20. The method of claim 19, wherein at least one of:
a. said fluid sample is a plurality of fluid samples comprising fluid samples from both subjects that suffer from GVHD and subjects that do not suffer from GVHD;
b. said cfDNA quantities comprise total cfDNA quantities in said samples and said at least one clinical parameter comprises ALT level; and
c. said cfDNA quantities comprise total cfDNA quantities and cfDNA quantities from monocytes in said samples and said at least one clinical parameter comprises ALT level.


SEQUENCE LISTING (56 sequences)

SEQ ID NO: 1 - DNA, 25 bp, synthetic construct
  source 1..25: mol_type = other DNA
tagtgttaggaatttaagggttagt

SEQ ID NO: 2 - DNA, 23 bp, synthetic construct
  source 1..23: mol_type = other DNA
ccaaaattaacaaaaacaaaaaa

SEQ ID NO: 3 - DNA, 28 bp, synthetic construct
  source 1..28: mol_type = other DNA
tgtattttaggtttttatatttttatta

SEQ ID NO: 4 - DNA, 25 bp, synthetic construct
  source 1..25: mol_type = other DNA
aaacaaaactactatcaaatccttc

SEQ ID NO: 5 - DNA, 27 bp, synthetic construct
  source 1..27: mol_type = other DNA
ttgttagaagtagttaaattatttttt

SEQ ID NO: 6 - DNA, 20 bp, synthetic construct
  source 1..20: mol_type = other DNA
ctaacccccaaatacaaacc

SEQ ID NO: 7 - DNA, 24 bp, synthetic construct
  source 1..24: mol_type = other DNA
ggtttgtgattttgtaagtattaa

SEQ ID NO: 8 - DNA, 21 bp, synthetic construct
  source 1..21: mol_type = other DNA
acccactatccctctaaactc

SEQ ID NO: 9 - DNA, 24 bp, synthetic construct
  source 1..24: mol_type = other DNA
attattagggagaggaaatatgtt

SEQ ID NO: 10 - DNA, 20 bp, synthetic construct
  source 1..20: mol_type = other DNA
ctaacccaccaaactccaaa

SEQ ID NO: 11 - DNA, 21 bp, synthetic construct
  source 1..21: mol_type = other DNA
tgggtgttgttattttgttga

SEQ ID NO: 12 - DNA, 21 bp, synthetic construct
  source 1..21: mol_type = other DNA
ctacaaaaatacacaccccaa

SEQ ID NO: 13 - DNA, 25 bp, synthetic construct
  source 1..25: mol_type = other DNA
atagtgaagatgttagtttgttttt

SEQ ID NO: 14 - DNA, 24 bp, synthetic construct
  source 1..24: mol_type = other DNA
aacacacttacctaataaccaaac

SEQ ID NO: 15 - DNA, 22 bp, synthetic construct
  source 1..22: mol_type = other DNA
ggtattttgaagaggtaggttt

SEQ ID NO: 16 - DNA, 21 bp, synthetic construct
  source 1..21: mol_type = other DNA
acctaaataccccaaactcat

SEQ ID NO: 17 - DNA, 27 bp, synthetic construct
  source 1..27: mol_type = other DNA
agtttttttataatagttttttgttat

SEQ ID NO: 18 - DNA, 24 bp, synthetic construct
  source 1..24: mol_type = other DNA
acactaaaatttcaaacaaaactc

SEQ ID NO: 19 - DNA, 26 bp, synthetic construct
  source 1..26: mol_type = other DNA
ttttttattgttttaatgttttttag

SEQ ID NO: 20 - DNA, 25 bp, synthetic construct
  source 1..25: mol_type = other DNA
taaactcaatcccctaaatatctac

SEQ ID NO: 21 - DNA, 23 bp, synthetic construct
  source 1..23: mol_type = other DNA
ggtattggggatattttagagag

SEQ ID NO: 22 - DNA, 22 bp, synthetic construct
  source 1..22: mol_type = other DNA
cctcctaaacaactcaccctac

SEQ ID NO: 23 - DNA, 22 bp, synthetic construct
  source 1..22: mol_type = other DNA
agtaaaatgttgggtaggtttt

SEQ ID NO: 24 - DNA, 25 bp, synthetic construct
  source 1..25: mol_type = other DNA
taaaatcatcttaaaaatcaaatac

SEQ ID NO: 25 - DNA, 25 bp, synthetic construct
  source 1..25: mol_type = other DNA
ttgtgttagtagagagaggaaagag

SEQ ID NO: 26 - DNA, 25 bp, synthetic construct
  source 1..25: mol_type = other DNA
cttcaaaatacaaaacactcatctc

SEQ ID NO: 27 - DNA, 20 bp, synthetic construct
  source 1..20: mol_type = other DNA
ggaggttaaggagagggagt

SEQ ID NO: 28 - DNA, 22 bp, synthetic construct
  source 1..22: mol_type = other DNA
aaaaaaaaactaccaaaacacc

SEQ ID NO: 29 - DNA, 21 bp, synthetic construct
  source 1..21: mol_type = other DNA
agtttggtttggggtttagag

SEQ ID NO: 30 - DNA, 21 bp, synthetic construct
  source 1..21: mol_type = other DNA
cacaccctctcacacaaacac

SEQ ID NO: 31 - DNA, 27 bp, synthetic construct
  source 1..27: mol_type = other DNA
gttagaaggtatagaaataattgttat

SEQ ID NO: 32 - DNA, 21 bp, synthetic construct
  source 1..21: mol_type = other DNA
tctccaaactctaaaaaccct

SEQ ID NO: 33 - DNA, 22 bp, synthetic construct
  source 1..22: mol_type = other DNA
gttttttgtttttgtaggttga

SEQ ID NO: 34 - DNA, 24 bp, synthetic construct
  source 1..24: mol_type = other DNA
cttcaaaatacaaaacactcatct

SEQ ID NO: 35 - DNA, 23 bp, synthetic construct
  source 1..23: mol_type = other DNA
ttttttattttttgggtatttgt

SEQ ID NO: 36 - DNA, 24 bp, synthetic construct
  source 1..24: mol_type = other DNA
taaaaacactcacattccaataaa

SEQ ID NO: 37 - DNA, 25 bp, synthetic construct
  source 1..25: mol_type = other DNA
ggtatagtgattagggggtagttat

SEQ ID NO: 38 - DNA, 24 bp, synthetic construct
  source 1..24: mol_type = other DNA
aaaataactaaaacaaaccctacc

SEQ ID NO: 39 - DNA, 24 bp, synthetic construct
  source 1..24: mol_type = other DNA
gagaagttaggaggagagtagata

SEQ ID NO: 40 - DNA, 22 bp, synthetic construct
  source 1..22: mol_type = other DNA
tttacattttaaattttatccc

SEQ ID NO: 41 - DNA, 26 bp, synthetic construct
  source 1..26: mol_type = other DNA
tttgtgtgaatagaaagattttagtt

SEQ ID NO: 42 - DNA, 22 bp, synthetic construct
  source 1..22: mol_type = other DNA
aatataactccaccccaaaatc

SEQ ID NO: 43 - DNA, 23 bp, synthetic construct
  source 1..23: mol_type = other DNA
tgatagatgaggaaattgaggtt

SEQ ID NO: 44 - DNA, 26 bp, synthetic construct
  source 1..26: mol_type = other DNA
aaaacaaactttaaaatctaatcaac

SEQ ID NO: 45 - DNA, 22 bp, synthetic construct
  source 1..22: mol_type = other DNA
ggaagttttggtatgatttttt

SEQ ID NO: 46 - DNA, 25 bp, synthetic construct
  source 1..25: mol_type = other DNA
actctaatataaacacctaacaacc

SEQ ID NO: 47 - DNA, 20 bp, synthetic construct
  source 1..20: mol_type = other DNA
ttttttttgagatgggattt

SEQ ID NO: 48 - DNA, 25 bp, synthetic construct
  source 1..25: mol_type = other DNA
taaaaaattaaaattacaataaacc

SEQ ID NO: 49 - DNA, 23 bp, synthetic construct
  source 1..23: mol_type = other DNA
gtagttgggatttagagaaggtt

SEQ ID NO: 50 - DNA, 22 bp, synthetic construct
  source 1..22: mol_type = other DNA
aacccacaacctaaaatcctac

SEQ ID NO: 51 - DNA, 20 bp, synthetic construct
  source 1..20: mol_type = other DNA
ggaattttggaggttgtagg

SEQ ID NO: 52 - DNA, 26 bp, synthetic construct
  source 1..26: mol_type = other DNA
ttatcttactaatcatactacttccc

SEQ ID NO: 53 - DNA, 22 bp, synthetic construct
  source 1..22: mol_type = other DNA
ggggttttaatttagggtttag

SEQ ID NO: 54 - DNA, 25 bp, synthetic construct
  source 1..25: mol_type = other DNA
aattcacaaattattaacaaacacc

SEQ ID NO: 55 - DNA, 24 bp, synthetic construct
  source 1..24: mol_type = other DNA
tagagtattggtttgaagatttgt

SEQ ID NO: 56 - DNA, 23 bp, synthetic construct
  source 1..23: mol_type = other DNA
tatcacaaccacacataaacaac